(19) United States
(12) Patent Application Publication
(10) Pub. No.: US 2011/0165266 A1
(43) Pub. Date: Jul. 7, 2011

(54) SCREEN FOR INHIBITORS OF HIV REPLICATION

(76) Inventors: James W. Bruce, Lake Mills, WI (US); Paul G. Ahlquist, Madison, WI (US); John A.T. Young, San Diego, CA (US)

(21) Appl. No.: 12/989,875

(22) PCT Filed: Apr. 30, 2009
(86) PCT No.: PCT/US2009/042288

§ 371 Date: Mar. 17, 2011

Related U.S. Application Data
(60) Provisional application No. 61/049,985, filed on May 2, 2008.
(60) Provisional application No. 61/126,273, filed on May 2, 2008.

(51) Int. Cl. A61K 33/20; C12Q 1/70; A61K 31/085; A61P 31/18

(52) U.S. Cl. 424/662; 435/5; 514/718

(57) ABSTRACT

A method of screening test chemicals or compounds as inhibitors of HIV replication is disclosed. In one embodiment, the method comprises the step of determining whether the test chemical or compound is a sulfonation inhibitor. In another embodiment, the invention is a method of treating an HIV infected individual to reduce HIV replication comprising the step of treating the individual with an effective amount of sulfonation inhibitor.

### CROSS-REFERENCE TO RELATED APPLICATION

This application claims priority from U.S. provisional patent application Ser. No. 61/049,985 filed May 2, 2008 and U.S. provisional patent application Ser. No. 61/126,273 filed May 2, 2008, both of which are incorporated by reference in entirety for all purposes.

### STATEMENT REGARDING FEDERALLY SPONSORED RESEARCH OR DEVELOPMENT

This invention was made with United States government support awarded by the following agencies: NIH CA022443 and NIH AI072645. The United States government has certain rights in this invention.

### BACKGROUND OF THE INVENTION

The early steps of retrovirus replication leading up to provirus establishment are highly dependent on cellular processes and represent a time when the virus is particularly vulnerable to antivirals and host defense mechanisms However, the roles played by cellular factors are only partially understood.

The Retroviridae are a large viral family that includes the human pathogens Human Immunodeficiency Viruses 1 and 2 (HIV-1 and HIV-2), the causative agents of acquired immune deficiency syndrome (AIDS). Due to their small coding capacity and requirement for integration into the host cell genome, retroviruses are heavily dependent upon host cell machinery for efficient replication.

The retroviral lifecycle can be divided into two distinct phases. The early stage consists of virus binding to a cellular receptor, fusion of viral and cellular membranes leading to delivery of the viral core into the cytoplasm, reverse transcription of the positive strand RNA genome to generate a double stranded DNA (dsDNA) product, translocation of viral nucleoprotein complexes to the nucleus, and provirus establishment through integration of the viral DNA into the host cell genome. The late stage consists of transcription of the viral genome by host RNA polymerase II (RNA pol II), RNA processing and export to the cytoplasm, translation of viral proteins, viral assembly, egress and maturation.

While progress has been made on the identification of many of the cellular proteins involved in the late stage of the retroviral lifecycle, particularly in transcription, RNA processing and egress, less is known about the contribution of cellular factors to the early stage of the retroviral lifecycle. In particular, the contribution of cellular factors to steps subsequent to virus:cell membrane fusion and that lead to proviral DNA establishment are only partially understood (Goff, Nature Rev. Microbiol. 5:253-263, 2007). A number of cellular factors that facilitate early steps in infection have been identified, although in some cases the roles of these factors are controversial.

These factors include the actin cytoskeleton and microtubule network (Arhel et al., Nature Methods 3:817-824, 2006; Bukrinskaya et al., J. Exp. Med. 188:2113-2125, 1998; Campbell et al., J. Virology 78:5745-5755, 2004; Leung et al., EMBO. J. 25:2155-2166, 2006; McDonald et al., J. Cell Biol. 159:441-452, 2002; Naghavi et al., EMBO. J. 26:41-52, 2007), LAP-2α barrier-to-autointegration factor (BAF), emerin (Chen et al., Proc. Nat'l Acad. Sci. USA 95:15270-15274, 1998; Jacque et al., Nature 441:641-645, 2006; Lee et al., Proc. Nat'l Acad. Sci. USA 95:1528-1533, 1998; Lin et al., J. Virology 77:5030-5036, 2003; Mansharamani et al., J. Virology 77:13084-13092, 2003; Shun et al., J. Virology 81:166-172, 2007; Suzuki et al., J. Virology 76:12376-12380, 2002; Suzuki et al., EMBO. J. 23:4670-4678, 2004), SUMOylation factors (Yueh et al., J. Virology 80:342-352, 2006), importins (Brass et al., Science 319:921-926, 2008; Fassati et al., EMBO. J. 22:3675-3685, 2003; Zielske et al., J. Virology 79:11541-11546, 2005), tRNAs (Zaitseva et al., PLoS Biol. 4:e332, 2006) and LEDGF (Cherepanov et al., J. Biol. Chem. 278:372-381, 2003; Emiliani et al., J. Biol. Chem. 280:25517-25523, 2005; Hombrouck et al., PLoS Pathog. 3:e47, 2007; Llano et al., J. Biol. Chem. 279:55570-55577, 2004; Llano et al., Science 314:461-464, 2006; Llano et al., J. Virology 78:9524-9537, 2004; Maertens et al., J. Biol. Chem. 278:33528-33539, 2003; Shun et al., Genes Dev. 21:1767-1778, 2007).

Although a recent genome-wide small interfering RNA (siRNA) screen uncovered a number of cellular genes that contribute to various stages of HIV infection, it was notable that only a few additional factors were described that are associated with either viral DNA synthesis or integration (Brass et al., Science 319:921-926, 2008). It therefore seems likely that other, as yet unidentified, cellular factors participate in early retroviral replication. Accordingly, there is a need for a method of screening test chemicals as inhibitors of HIV replication.

### SUMMARY OF THE INVENTION

The present invention is a method of screening inhibitors of HIV replication. It relies on the inventors' observations that the host cell sulfonation pathway influences retroviral infection.

In a first aspect, the present invention is a method of screening test agents as inhibitors of HIV replication. In one embodiment of the first aspect, the method comprises the step of (a) determining whether the test agent is a sulfonation inhibitor, wherein if the test agent is a sulfonation inhibitor, then the test agent is a suitable inhibitor of HIV replication.

In different embodiments of the first aspect, the method also comprises the step of (b) determining whether the test agent is an inhibitor of PAPSS1 or (b) determining whether the test agent is an inhibitor of at least one sulfotransferase.

In a second aspect, the present invention is a method of screening test chemicals or compounds for inhibition of HIV replication. In one embodiment of the first aspect, the method comprises the steps of (a) exposing a test chemical or compound to a cell; (b) exposing a first and a second HIV vector to the cell, wherein the first HIV vector is sensitive to the cell's sulfonation pathway and the second HIV vector is insensitive to the cell's sulfonation pathway and wherein both HIV vectors comprise genes encoding reporter molecules; and (c) examining the results of steps (a) and (b), wherein a test chemical or compound that interferes with reporter gene expression from the first, but not the second, HIV vector, is a suitable inhibitor of HIV replication.

In different embodiments of the second aspect, the test chemical interferes with the function of a sulfonation-regulated effector of HIV gene expression or the cell is a mammalian cell, a HEK293 cell, a Jurkat cell, another human T-cell line, THP-1, other human macrophage/monocyte cell line, primary T lymphocyte, or primary macrophage/monocyte. In other embodiments of the second aspect, the reporter gene of the sulfonation insensitive vector is β-galactosidase, and the reporter gene of the sulfonation sensitive vector is luciferase. The first and second HIV vectors are pseudotyped with the vesicular stomatitis virus glycoprotein, and the expression of reporters is measured by chemiluminescent assay. The sulfonation insensitive vector is PLenti6/V5-GW/lacZ, and the sulfonation sensitive vector is pNL4-3.Luc.R-E-. In further embodiments of the second aspect, the inhibitor is an inhibitor of PAPSS1 or an inhibitor of at least one sulfotransferase, the first and second HIV vectors are exposed to the cells sequentially on replicate plates, or the first and second HIV vectors are exposed to the cells concurrently.

In various embodiments of the second aspect, the method also comprises the step of (d) exposing an third and fourth retroviral vector to the cell, wherein the third vector has LTRs that are sensitive to sulfonation pathway inhibition and the fourth retroviral vector is insensitive to sulfonation pathway inhibition and wherein both the third and fourth retroviral vectors comprise genes encoding reporter molecules. In still other embodiments of the second aspect, the third retroviral vector is selected from the group comprising HIV and murine leukosis virus (MLV) and the fourth retroviral vector is selected from the group comprising avian sarcoma and leukosis virus (ASLV).

In a third aspect, the present invention is a method of treating an HIV infected individual to reduce HIV replication comprising the step of treating the individual with an effective amount of sulfonation inhibitor.

In a fourth aspect, the present invention is a method of screening test chemicals for inhibition of HIV replication, comprising the steps of (a) exposing a test chemical to a cell; (b) exposing an first and second retroviral vector to the cell, wherein the first vector has LTRs that are sensitive to sulfonation pathway inhibition and the second vector has LTRs that are not sensitive to sulfonation pathway inhibition and wherein both the first and second vectors comprise genes encoding reporter molecules; and (c) examining the result of steps (a) and (b), wherein a test compound that interferes with reporter gene expression from the first vector, but not the second vector is a suitable inhibitor of HIV replication.

In one embodiment of the fourth aspect, the first retroviral vector is selected from the group comprising HIV and MLV, and the second retroviral vector is selected from the group comprising ASLV.

In a final aspect, the present invention is a method of screening test chemicals for inhibition of HIV replication, comprising the steps of (a) exposing a test chemical or compounds to a cell; (b) exposing a first and a second HIV vector to the cell, wherein the first HIV vector is sensitive to the cell's sulfonation pathway and the second HIV vector is insensitive to the cell's sulfonation pathway and wherein both HIV vectors comprise genes encoding reporter molecules; and (c) examining the result of steps (a) and (b), wherein a test chemical or compound that interferes with HIV expression either by blocking sulfonation pathway components or cellular processes regulated by sulfonation is a suitable inhibitor of HIV replication.

Other objects, advantages and features of the present invention will become apparent after review of the specification, claims and drawings.

### BRIEF DESCRIPTION OF THE DRAWINGS

FIG. 1. Scheme used to isolate pRET-mutagenized Chinese hamster ovary (CHO-K1) cell lines that are resistant to subsequent retroviral infection. CHO-K1 cells were mutagenized by infection with a vesicular stomatitis virus glycoprotein (VSV-G) pseudotyped pRET vector at a multiplicity of infection (moi) of 0.01 G418^{R} transducing units and selected in G418 for 2 weeks. Pools of mutagenized cells (1×10⁷) were challenged with a VSV-G pseudotyped murine leukemia virus (MLV) vector that encodes CD4. Infected cells were depleted from the population by magnetic sorting with an iron conjugated anti-CD4 antibody. After five rounds of challenge and sorting, the enriched pools were infected with a VSV-G pseudotyped MLV vector that encodes the red fluorescent protein HcRed. The non-fluorescent cells were single cell cloned by fluorescent activated cell sorting (FACS), expanded and seeded into duplicate assay plates. The assay plates were infected with another VSV-G pseudotyped MLV vector that encodes β-galactosidase. One plate was then assayed with a chemiluminescent assay for β-galactosidase. For control purposes, the other plate was assayed with a luciferase based chemiluminescent assay to measure viable cell number.

FIG. 2. Resistance of the IM2 cell line maps to the MLV core. (FIG. 2A) CHO-K1 and IM2 cells were challenged with serial dilutions of the VSV-G pseudotyped MLV vector (MMP-nls-lacZ[VSV-G]), encoding β-galactosidase. The cells were then stained 48 hpi with X-gal, the number of blue cells were counted, and the data reported as the percentage of LacZ transducing units (LTU) obtained from wild-type (WT) CHO-K1 infections (5×10⁵ LTU). The data shown are the average of three experiments each performed with triplicate samples. Error bars indicate the standard deviation of the data. (FIG. 2B) CHO-K1 cells and IM2 cells, engineered to express TVA800, or WT CHO-K1 cells were challenged with either pMMp-nls-LacZ[envA], an EnvA pseudotyped MLV vector encoding β-galactosidase, or with RCASBP(A)-AP, a subgroup A avian sarcoma and leukosis virus (ASLV-A) vector encoding heat stable alkaline phosphatase. Infection was monitored using chemiluminescent assays to detect reporter enzyme activities along with a chemiluminescent assay to measure relative viable cell numbers. The ratios of [enzyme activities:relative viable cell number] were calculated for each sample and compared with values from CHO-K1 TVA 800 cells (defined as 100% infection). The data shown are the average mean values obtained in an experiment performed with quadruplicate samples and are representative of three independent experiments. Error bars indicate the standard deviation of the data.

FIG. 3. PAPS synthase 1 (PAPSS1) gene is disrupted in IM2 cells. (FIG. 3A) The pRET mutagenic vector is integrated upstream of exon 12 of the PAPSS1 gene. The nucleotide sequence of the fusion junction that is formed via mRNA splicing involving the splice donor located downstream of the NPT gene in pRET, and the splice acceptor located upstream of exon 12 of the hamster PAPSS1 gene is shown. Since the hamster PAPSS1 gene has not been previously characterized by DNA sequencing, this sequence at the fusion junction is shown aligned with that of the corresponding mouse PAPSS1 exon 11-exon 12 junction nucleotide sequence. The amino acid sequence encoded by this region of mouse PAPSS1 is also shown. The nucleotide differences between the hamster and mouse sequences occur at codon wobble positions (indicated with lowercase letters) that do not alter the amino acid sequence. (FIG. 3B) IM2 cells were engineered to express human PAPSS1 or PAPSS2, or a control cDNA, as described in Example I, Materials and Methods. Lysates prepared from these cells were assayed for PAPSS activity by adding ATP and ^{[35]}S labeled sulfate, and the samples were then subjected to thin layer chromatography to separate the substrate from the reaction products (APS and PAPS). The experiment shown is representative of three independent experiments. (FIG. 3C) The amounts of PAPS synthesized in the samples shown in FIG. 3C were quantitated as described in Example 1, Materials and Methods, and are shown relative to the amount produced by the WT CHO-K1 cell extract (defined as 100%). The data shown are the average of three independent experiments. Error bars indicate the standard deviation of the data.

FIG. 4. PAPSS activity is required for efficient MLV infection. (FIG. 4A) CHO-K1 and IM2 cells engineered to express either a control complementary DNA (cDNA), human PAPSS1 or human PAPSS2 were challenged with serial dilutions of the VSV-G pseudotyped MLV vector (MMP-nls-lacZ[VSV-G]), encoding β-galactosidase. The cells were then stained 48 hours post infection (hpi) with X-gal, the number of blue cells were counted, and the data reported as the percentage of LacZ transducing units (LTU) obtained from WT CHO-K1 infections (5×10⁵ LTU). The data shown are the average mean values obtained with triplicate samples. (FIGS. 4B and 4C) CHO-K1 cells were challenged with a VSV-G pseudotyped MLV vector MMP-nls-lacZ[VSV-G]) in the presence of either 100 mM chlorate, 5 mM Guaiacol, or both 100 mM chlorate and 5 mM Guaiacol. The cells were subsequently assayed for either β-galactosidase activity using a chemiluminescent assay (FIG. 4B), or for viable cell number using a chemiluminescent assay (FIG. 4C). The data in FIGS. 4B and 4C are the average mean values obtained in an experiment performed with quadruplicate samples and are reported as a percentage of that seen with untreated cells. (FIG. 4D) Chicken DF-1 cells were challenged with either the MLV vector pMMp-nls-LacZ[VSV-G], or the ASLV-A vector RCASBP(A)-AP in the presence of 100 mM chlorate and assayed 48 hpi with chemiluminescent assays for reporter enzyme activities or viable cell number. The ratios of [enzyme activities:relative viable cell number] were calculated for each sample and compared with untreated controls (defined as 100% infection). The data shown are the average mean values obtained in an experiment performed with quadruplicate samples. The results shown in FIGS. 4A-4D are representative of three independent experiments and error bars indicate the standard deviation of the data.

FIG. 5. The sulfonation pathway does not influence reverse transcription or the level of integrated virus DNA. (FIGS. 5A and 5B) CHO-K1 cell lines were challenged with the VSV-G pseudotyped MLV vector encoding the enhanced green fluorescent protein (LEGFP). Total DNA (FIG. 5A) or DNA from isolated nuclei (FIG. 5B) were harvested at 0 or 24 hpi, DNA concentration was quantitated by A260, and a real-time (PCR) amplification analysis was performed to measure the levels of viral DNA that were synthesized. (FIG. 5C) Untreated CHO-K1 cells or CHO-K1 cells that had been pretreated for 16 hrs with 100 mM chlorate and maintained in medium containing this concentration of chlorate were challenged with the MLV vector and subsequently analyzed for reverse transcription products as described in FIG. 5A. (FIG. 5D) CHO-K1 cells that were either untreated or treated with 100 mM chlorate, IM2 cells, and MCL7 cells, were challenged with the same VSV-G pseudotyped MLV vector and total DNA was harvested at 1 or 18 days post infection for real time PCR quantitation of viral DNA products. The chemically-mutagenized MCL7 cell line displays a strong block to MLV DNA integration (Bruce et al., J. Virology 79: 12969-12978, 2005). Chlorate treated CHO-K1 cells were passaged in medium containing 100 mM chlorate for the duration of the experiment. The data shown in FIGS. 5A-5D are the average mean values obtained in independent experiments performed with triplicate samples and each is representative of three independent experiments. Error bars indicate the standard deviation of the data.

FIG. 6. The sulfonation pathway influences transcription specifically from the MLV long terminal repeat (LTR). (FIG. 6A) CHO-K1 or IM2 cells were challenged with the VSV-G pseudotyped MLV vectors MMP-nls-lacZ, which directs MLV LTR-driven lacZ gene expression, or with pQCLIN, a self-inactivating MLV vector with defective LTRs and an internal cytomegalovirus (CMV) promoter which drives lacZ expression. The cells were subsequently assayed for β-galactosidase activity and viable cell number and the data reported as in FIG. 2B with the ratio of [β-galactosidase activity:relative viable cell number] observed with CHO-K1 cells defined as 100% infection. (FIG. 6B) CHO-K1 cells were challenged with the same virus vectors used in FIG. 5A but in the presence of 100 mM chlorate and 5 mM guaiacol. The cells were subsequently assayed for β-galactosidase activity as in FIG. 6A. The data shown in FIGS. 6A and 6B are the average mean values obtained in an experiment performed with quadruplicate samples and each is representative of three independent experiments. (FIG. 6C) Total RNA was isolated from CHO-K1 or IM2 cells that had been infected with the VSV-G pseudotyped MLV vector pCMMP-EGFP. Ribonuclease (RNase) protection assays were performed using a probe that recognizes either the provirus-derived transcript or the hamster β-actin gene. Protected fragments were separated, subjected to gel electrophoresis as described in Example 1, Materials and Methods, and exposed to a phosphoimager plate. (FIG. 6D) CHO-K1 cells were challenged with virus as in FIG. 6C in the presence of either 100 mM chlorate, 5 mM guaiacol, or with both inhibitors. The inhibitors were present throughout the infection. (FIG. 6E) The mean average data of at least three independent RNase protection experiments, conducted as in FIGS. 6C and 6D, were quantitated by phosphorimaging using the image quant software volume method. The relative levels of viral transcripts were normalized to the corresponding β-actin levels and are reported as a percentage of those seen with untreated CHO-K1 cells (defined as 100%). Error bars in FIGS. 6A, 6B and 6E, indicate the standard deviation of the data.

FIG. 7. Chlorate treatment during virus infection reduces reporter gene expression from newly acquired, but not resident proviruses. (FIG. 7A) CHO-K1 cells (1×10⁵) were challenged with 5×10⁵ IU of MMP-nls-lacZ[VSV-G] at 4° C. for 2 hrs and then warmed to 37° C. to initiate infection at t=0 mins. Chlorate was added to 100 mM final concentration at the indicated hpi. At 48 hpi, the cells were assayed for β-galactosidase activity and for relative viable cell numbers and the level of infection was calculated as in FIG. 2B. The data shown are the average mean values obtained in an experiment performed with triplicate samples. (FIG. 7B) Cells harboring a resident MLV provirus, pCMMP-SEAP, encoding secreted alkaline phosphatase, were challenged with a second MLV vector, MMP-nls-lacZ[VSV-G] encoding β-galactosidase, in the presence of 100 mM chlorate. Chemiluminescent assays were then used to measure reporter enzyme activity levels (FIG. 7B), as well as relative viable cell numbers (FIG. 7C), and the values obtained with untreated cells was defined as 100% in each case. The data shown in FIGS. 7C and 7D are the average mean values obtained in an experiment performed with quadruplicate samples. The data in FIGS. 2A-2C are representative of three independent experiments and error bars indicate the standard deviation of the data.

FIG. 8. PAPSS activity affects transcription from the HIV-1 LTR. (FIG. 8A) CHO-K1 or IM2 cells were challenged with either one of two VSV-G pseudotyped HIV-1 vectors, NL43E-R-Luc, that directs luciferase gene transcription from the viral LTR, or with pLenti6/V5-GW/LacZ, a self inactivating HIV-1 vector from which β-galactosidase expression is driven by an internal CMV promoter. Chemiluminescent assays were used to measure reporter enzyme activities and viable cell numbers. The data shown was calculated as in FIG. 2B and the value obtained with CHO-K1 cells was defined as 100% infection. (FIG. 8B) CHO-K1 cells were challenged with the viruses described in FIG. 8A, in the presence of either 100 mM chlorate, 5 mM Guaiacol or both inhibitors and subsequently assayed as in FIG. 8A. (FIG. 8C) Jurkat cells were spin inoculated with the VSV-G pseudotyped vectors NL43E-R-Luc[VSV-G] or MLV-LUC (an MLV vector that encodes luciferase) in the presence of 120 mM chlorate, 5 mM guaiacol or both inhibitors. Chemiluminescent assays were used to monitor virus infection as in FIG. 8A. The viable cell number observed in the experiment shown in FIG. 8C is reported in FIG. 8D. For FIGS. 8B-8D the data are reported as the percentage of untreated controls. For FIGS. 8A-8D, the data is the average mean values obtained in an experiment with quadruplicate samples and are representative of three independent experiments. Error bars indicate the standard deviation of the data.

FIG. 9. Schematic diagrams of the proviral forms of MLV vectors used in this report. (FIG. 9A) Following integration of the pRET vector in a reverse orientation within an intron of a cellular gene, messenger RNA (mRNA) splicing gives rise to an IRES-containing transcript that encodes GFP. An internal promoter drives expression of the neomycin phosphotransferase gene (NPT) which confers G418 resistance only when a downstream mRNA instability motif is removed by splicing to a downstream cellular exon. A downstream poly (A) signal derived from the cellular gene is captured to stabilize the RNA. (FIG. 9B) The MLV pCMMP based vectors have the gag/pol and env genes replaced with various reporter genes, including CD4, HcRed, LacZ, and luciferase. Reporter gene expression is driven from the viral LTR. The MLV vector pLEGFP has a similar structure but has both WT LTRs and an internal CMV promoter driving GFP expression. (FIG. 9C) The self-inactivating MLV vector pQLIN has the U3 elements of the viral LTRs deleted and the gag/pol and env genes replaced with the CMV promoter driving expression of the LacZ gene.

FIG. 10. Total sulfonation of macromolecules is reduced in IM2 cells. CHO-K1 and IM2 cells were incubated in sulfate free media supplemented with 200 μCi/ml ^{[35]}SO₄ for 48 hours. Cells were then harvested in 50 mM Tris-pH 7.0, 2% SDS. The samples were then precipitated in 25% TCA. The precipitates were washed 3× with 5% TCA, once with 95% ethanol and air dried. Samples were suspended in scintillation fluid and counted for 1 minute, and incorporation of label was normalized to CHO-K1 values. Values shown are the average of quadruplicate sample readings from two independent labeling experiments. Error bars indicate the standard deviation of the data.

FIG. 11. Effect of chlorate on MLV vector infection of IM2 cells. IM2 cells were challenged with the MLV vector pMMp-nls-LacZ[VSV-G] in the presence of 120 mM chlorate and assayed 48 hpi with chemiluminescent assays for reporter enzyme activities or viable cell number. The ratios of [enzyme activities:relative viable cell number] were calculated for each sample and compared with untreated controls (defined as 100% infection). The data shown are the average mean values obtained in an experiment performed with quadruplicate samples. The results are representative of three independent experiments and error bars indicate the standard deviation of the data.

FIG. 12. (FIG. 12A) Standard curve of real-time quantitative PCR analysis. Serial dilutions of pLEGFP-C1 plasmid were amplified as described in materials and methods and the threshold Cycle value for each dilution was plotted against the number of input molecules of DNA. Non-linear regression analysis was performed on the data and the r² value was used to determine the fit of the data. The data in FIG. 12A was used to generate the standard curve for the results shown in FIG. 5D. (FIG. 12B) To determine if the QPCR assay was linear under the conditions of our analysis, 1×10⁶ cells were infected at an moi of 10 (10 times higher than the amounts used in our standard assay conditions as described in FIG. 5). Total DNA was isolated and then real time PCR analysis was performed as described in Example 1, Materials and Methods using the indicated dilutions of input viral DNA (used as a surrogate marker of the number of virions added). The data shown in are the average mean values obtained in independent experiments performed with triplicate samples and each is representative of three independent experiments. Error bars indicate the standard deviation of the data.

FIG. 13. Screening strategy for sulfonation dependent inhibitors of HIV. 293 cells will be treated with chemicals at 10 μM. Cells will then be infected with a sulfonation sensitive HIV variant to identify compounds that inhibit HIV infection. Antiviral compounds will then be counterscreened with a sulfonation insensitive variant of HIV to rule out sulfonation independent hits such as cell viability.

FIG. 14. Screen performed to identify chemical inhibitors of sulfonation dependent HIV infection. A primary screen of 18,976 compounds using a sulfonation sensitive virus identified 1,850 compounds with antiviral activity. Secondary screening with sulfonation sensitive HIV and MLV, as well as counterscreens with sulfonation insensitive HIV and cell viability identified 19 compounds with potential sulfonation dependent anti-retroviral activity.

FIG. 15. Test of inhibitory compounds' effects on sulfonation sensitive HIV. The effects of the 19 compounds from the primary screen on sulfonation sensitive HIV were tested across a wide concentration range. For display purposes, only the 5 μM concentration is shown. All compounds showed at least some inhibition of sulfonation sensitive HIV at this concentration with compounds 1, 4, 5, 7, and 14 inhibiting greater than 70%. Compounds 4 and 14 (grey) showed significant inhibition of sulfonation sensitive HIV and MLV, but not insensitive HIV (see also FIG. 16-17). These compounds also had limited effects on cell viability (FIG. 18). Compound 7 inhibited HIV in a sulfonation independent manner and had negative effects on cell viability see (FIG. 16-18).

FIG. 16. Test of inhibitory compounds' effects on sulfonation insensitive HIV. The effects of the 19 compounds from the primary screen on sulfonation sensitive HIV were tested across a wide concentration range. For display purposes, only the 5 μM concentration is shown. Most compounds showed little inhibition of sulfonation insensitive HIV at this concentration with the exception of compounds 1, 5, 6, 7, and 9 inhibiting greater than 25%.

FIG. 17. Test of inhibitory compounds' effects on sulfonation insensitive MLV. The effects of the 19 compounds from the primary screen on sulfonation sensitive HIV were tested across a wide concentration range. For display purposes, only the 5 μM concentration is shown. All compounds showed at least some inhibition of sulfonation sensitive MLV at this concentration with compounds 1, 4, 5, 7, 9, and 18 inhibiting greater than 70%. Compound 14 inhibited MLV more than 70% at 10 μM.

FIG. 18. Test of inhibitory compounds' effects on cell viability. The effects of the 19 compounds from the primary screen on sulfonation sensitive HIV were tested across a wide concentration range. For display purposes, only the 5 μM concentration is shown. Most compounds have minor effects on cell viability at this concentration with the exception of compounds 5, 7, and 18.

FIG. 19. Compounds 4 and 14 preferentially inhibit expression from newly acquired provirus. HEK 293 cells containing a previously established MLV (FIG. 19A) or HIV (FIG. 19B) provirus expressing the secreted gaussia luciferase gene were challenged with a second MLV (FIG. 19A) or HIV (FIG. 19B) vector encoding firefly luciferase in the presence of either compounds 4 or 14. 48 hours post infection reporter assays for firefly and gaussia luciferase were performed. Consistent with the effects of other sulfonation inhibitors, compounds 4 and 14 had a significantly greater inhibitory effect on newly acquired proviral expression than on resident proviral expression.

FIG. 20. Compounds 4 and 14 inhibition of HIV infection is synergistic with low sulfate conditions. (FIG. 20A) Infection of cells with HIV is identical when cells are grown in either low or high sulfate media. (FIG. 20B) Inhibition by compounds 4 and 14 is significantly enhanced in low sulfate media, indicating that these compounds affect the sulfonation pathway. In contrast, the sulfonation independent reverse transcription inhibitor AZT is unaffected by low sulfate conditions. Similar synergistic effects were seen using suboptimal concentrations of the PAPS synthase inhibitor chlorate (see FIG. 21).

FIG. 21. Compounds 4 and 14 inhibition of HIV infection is synergistic with suboptimal concentrations of the PAPS synthase inhibitor chlorate. (FIG. 21A) Infection of cells with HIV is identical when cells are grown in either standard media or media supplemented with 20 mM chlorate. (FIG. 21B) Inhibition by compounds 4 and 14 is significantly enhanced in media containing 20 mM chlorate, indicating that these compounds affect the sulfonation pathway. In contrast, the sulfonation independent reverse transcription inhibitor AZT is unaffected by 20 mM chlorate.

FIG. 22. Compound 23 inhibits sulfonation sensitive HIV, but not MLV. Cells were treated with 10 μM Compound 23 and infected with either sulfonation sensitive (sHIV), insensitive (iHIV) vectors or sulfonation sensitive (sMLV), insensitive (iMLV) MLV vectors. The cells were assayed for luciferase 48 hours post infection. Viable cell number was assayed by cell titer glo (Promega). Only sulfonation sensitive HIV was inhibited under these conditions.

### DETAILED DESCRIPTION OF THE INVENTION

A. In General

To identify other cellular factors that are involved in the early steps of retrovirus replication leading up to provirus establishment, the inventors have employed a somatic cell mutagenesis-based approach. PLoS Pathog. 4(11):e1000207, 2008, a manuscript authored by the inventors of the present invention, discloses preferred embodiments of the present invention and is incorporated by reference herein. The manuscript describes the inventors' work in identifying cellular proteins that participate in the early stages of retroviral replication. The inventors describe a high volume screening of insertionally mutagenized somatic cells that led to the isolation of a clonal cell line exhibiting 10-fold resistance to MLV infection. The 3′-phosphoadenosine 5′-phosphosulfate synthase 1 (PAPSS1) was identified as the mutant gene in these cells responsible for viral resistance. The experiments disclosed in Example 1 confirm a role for the cellular sulfonation pathway in MLV and HIV infection using chlorate, an inhibitor of PAPSS enzymes.

In one embodiment, the present invention is a method of screening test agents as inhibitors of HIV replication comprising the step of determining whether the test agent is a sulfonation inhibitor. If the test agent is a sulfonation inhibitor, then the agent is a suitable inhibitor of HIV replication. Preferably the method additionally comprises the step of determining whether the test agent is an inhibitor of PAPSS1 and/or determining whether the test chemical is an inhibitor of at least one sulfotransferase.

In a second embodiment, the present invention is a method of screening test agents as inhibitors of a target (e.g. protein) that is regulated by sulfonation wherein the target regulates HIV gene expression. If the test agent inhibits a target that is regulated by sulfonation and the target regulates gene expression, then the agent is a suitable inhibitor of HIV replication. The primary screen will detect inhibitors that affect a component of the sulfonation pathway or block the activity of an effector that is regulated by the pathway.

In a preferred embodiment, the present invention is a method of screening test chemicals or compounds for inhibition of HIV replication, comprising the steps of exposing the test chemical or compound to a cell; exposing a first and a second HIV vector to the cell, wherein the first HIV vector is insensitive to the cell's sulfonation pathway and the second HIV vector is sensitive to the cell's sulfonation pathway and wherein both HIV vectors comprise genes encoding reporter molecules, and examining the result of steps (a) and (b), wherein a test compound that inhibits the gene expression of the second HIV vector and not the first HIV vector, as measured by expression of the reporters, is a suitable inhibitor of HIV replication.

In a preferred embodiment of the invention, the method of additionally comprising the step of exposing a first ASLV vector and a second MLV vector to the cell, wherein the ASLV vector is insensitive to the cell's sulfonation pathway and the MLV vector is sensitive to the cell's sulfonation pathway and wherein both the ASLV and MLV vectors comprise genes encoding reporter molecules.

In another embodiment, the present invention is a method of treating an HIV infected individual to reduce HIV replication comprising the step of treating the individual with an effective amount of sulfonation or sulfonation pathway inhibitor.

B. Test Agents, Chemicals, or Compounds

One embodiment of the present invention is a screen for inhibitors of the cellular sulfonation pathway. The identified inhibitors are expected to inhibit a step coincident with the provirus establishment of HIV in human cells and inhibit subsequent viral gene expression. Example 1 discloses chlorate and guaiacol as inhibitors.

One of skill in the art would understand that many different chemicals or compounds could be screened for inhibition of the cellular sulfonation pathway, including small molecules, natural products, peptides, and proteins. Also included would be nucleic acids, such as siRNAs, small hairpin RNAs (shRNAs), antisense oligonucleotides, and ribosymes.

As indicated in Example 1, the inventors conducted a primary screen of compounds in the Maybridge portfolio and known bioactive libraries. Other suitable groups of compounds would include the Chembridge collection (16,000 compounds), ChemDiv collection (20,000 compounds), and the NCI open collection (140,000 compounds). We mean the terms “agents,” “chemical,” and “compounds” to be interchangeable. All of the terms indicate a test substance that one would evaluate as a screen for HIV inhibition.

C. Cell Lines

In a preferred embodiment one would use HEK293 cells, a common human cell line used in chemical screens and retroviral studies, in 96-well arrays as targets for chemical treatments. One of skill in the art would understand that other human and mammalian cells could replace HEK293 cells. Any mammalian cell line that is able to be infected by HIV would be useful. Almost all human and most mammalian cell lines are able to be infected by VSV-G pseudotyped HIV vectors to some degree. One would do the screen in almost any easily cultured human cell line.

We envision that the screen or follow-up analysis could be preferred in T-cell lines (e.g. Jurkat and CEB) and monocytes/macrophage lines (e.g. U937 lines) to ensure that the chemicals or compounds had the same effect in a more HIV relevant line or under more WT infections. Other preferred cell types are primary lymphocytes and monocyte/macrophages.

Our initial screen utilized CHO-K1 cells, which are Chinese hamster ovary cells, because these are robust cells that are functionally haploid. At the time they were a good choice for screening mutagenized cells. They would be a suitable choice for the chemical screen described below. However, they would not be the best choice for the siRNA screen because the hamster genome has not been sequenced and no hamster siRNA library exists.

If one wishes to screen using replication-competent HIV-1, one would want to use human cells that express both CD4 and one or both of the major viral coreceptors (CCR5 or CXCR4).

D. Appropriate Vectors

In one preferred embodiment of the invention, we propose the use of two different HIV vectors modified to deliver reporter genes in the screen. The first is a sulfonation sensitive virus, preferably nearly WT in nature that packages a genome with the sulfonation sensitive viral LTR driving expression of a marker gene. The second is a sulfonation insensitive vector that packages a genome encoding a reporter gene from an internal promoter. Since we have mapped the effect of the sulfonation to the viral LTR promoter, neither the specifics of inactivating mutations in the packaged genome nor the method of complementing these defects in the producer cells will affect the screening methods. In one version of the screen, we will screen compounds by using WT replication competent HIV in the appropriate cell lines (e.g. Jurkats, CEB, or primary lymphocytes and monocytes). Preferably, both vectors will be pseudotyped with the vesicular stomatitis virus glycoprotein (VSV-G) to eliminate entry effects.

We use the word “vector” to denote a genetically engineered virus or WT virus. An “HIV vector” is therefore a genetically engineered HIV virus or WT virus. This genetic engineering could result in a WT particle, but the particle may be produced from transfection, producer cell lines, etc. A suitable vector can be a nearly wild-type vector that can infect the natural host and potentially cause disease or can be is highly attenuated and may contain only a few hundred bases of viral sequence that allow packaging into the viral capsid. Preferable HIV vectors all have several mutations in them that make them only able to support one round of infection. These include, at a minimum, the inactivation of the envelope gene, but most have much more severe mutations. These were used for experimentation. The viral core and accessory proteins must be provided, but they do not need to be encoded in the genome that gets packaged into the virus.

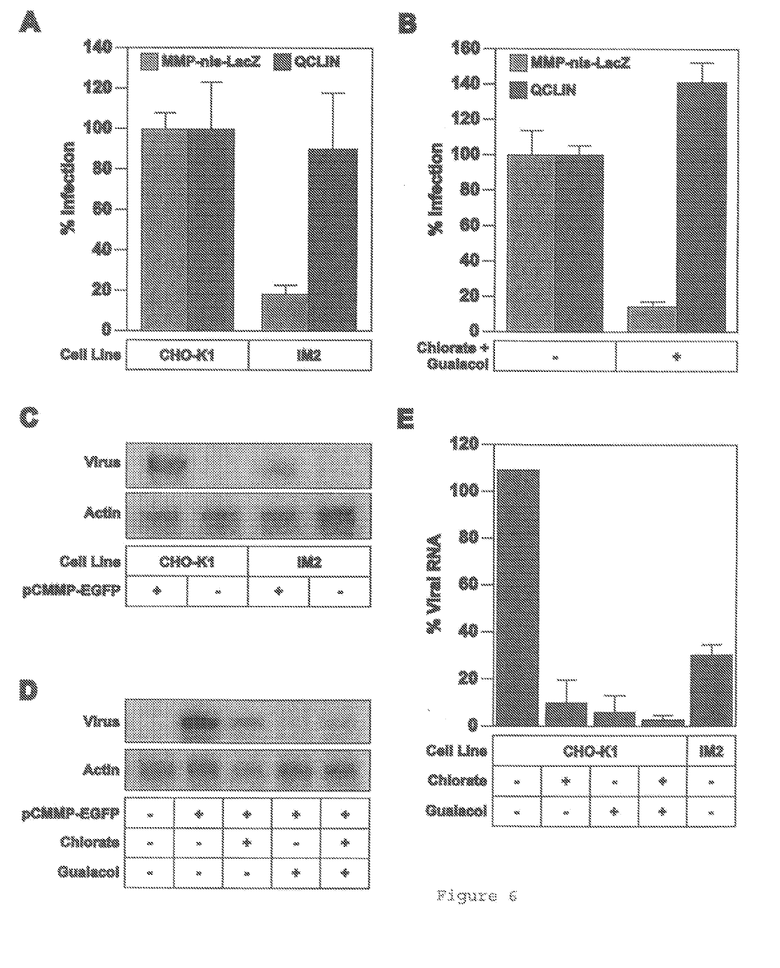

We envision using WT HIV in appropriate cell lines to test our claims, as well as genetically modified HIV vectors that contain all the HIV structural and replication components that allow for proper virion assembly, particle maturation, reverse transcription, and integration. These include the Gag gene, which makes capsid, nucleocapsid, and matrix, the Pol gene, which makes integrase and reverse transcription, and protease. These can be in cis in the viral genome or provided in trans by a producer cell line or by co-transfection of a plasmid. The genetically modified HIV vectors must also contain a suitable envelope protein for mediating viral entry provided either in cis or trans and a genome containing, at a minimum, viral sequences permitting efficient reverse transcription packaging, integration, and the sulfonation dependent or independent viral LTR driving gene expression. Preferably, the envelope protein is HIV gp160. More preferably, the envelope protein is VSV-G. Ideally, the genome would contain an easily assayable marker gene such as luciferase, but gene expression of both WT virus and these vectors could be followed by isolation of viral RNA and quantitation by either reverse transcription real time PCR or RNase protection assays (as done in FIGS. 6C, 6D, and 6E).

In a preferred embodiment, the first virus is a sulfonation sensitive HIV vector that drives expression of the luciferase reporter gene from the viral LTR promoter and the second would be a sulfonation-sensitive vector. A sulfonation sensitive vector has LTRs that are sensitive to sulfonation pathway inhibition. A sulfonation insensitive vector has LTRs that are not sensitive to inhibition of the sulfonation pathway. The sulfonation-sensitive HIV-1 vector is one that would contain a promoter element that is subject to transcriptional regulation by the sulfonation pathway. Currently, we envision that this would typically be the wild-type LTR and any derivatives (mutants) of that LTR that maintain the same mode of regulation. We envision that one may wish to use multiple envelope glycoprotein-receptor combinations. For example, retroviral vectors can be pseudotyped with a variety of other viral envelope glycoproteins that can use receptors that are either endogenously expressed in human cells or could be introduced into human cells so that they become a target for such a virus vector.

A preferred sulfonation sensitive virus is pNL43.Luc.R-.E-, which is available from the NIH AIDS Research and Reference Reagent Program, catalog #3418 and was contributed by Dr. Nathaniel Landau (Virology 206: 935-944, 1995). Any vector with a WT LTR in the natural position can be used to drive sulfonation dependant reporter gene expression.

As the sulfonation insensitive vector, one would preferably use pLenti6N5-GW/lacZ, which is a component of the Invitrogen VIRAPOWER pLenti6N5 Directional TOPO cloning Kit, catalog number K4955-10. Any vector that expresses a reporter gene from an internal promoter could be used. Note that the invention does not require a specific combination of reporter and vector. We have derivatives of the pLenti6 vector that express renilla luciferase, firefly luciferase SEAP and GFP and versions of pNL43 that we have constructed that express guassia luciferase and GFP. All are suitable.

A preferred second vector is a self-inactivating HIV vector with an internal CMV promoter driving expression of luciferase which was derived from the commercial vector pLVX-DsRed-Monomer-C1 (Clontech, catalog #632153). This virus is known to be insensitive to the sulfonation pathway and will serve as a control for sulfonation independent effects such as cytotoxicity or effects on VSV-G.

E. Screening

The screen will typically be performed by seeding multiple cells, typically 293 cells in 96-well plates (1×10⁴ cells/well), the day before the experiment. The cells will then physically be infected with at least 1×10⁴ transducing units of either a sulfonation sensitive or insensitive virus along with the test compound. The infections could be performed sequentially on replicate plates. Alternatively, the cells can be co-infected if different reporter genes (such as luciferase and β-galactosidase) are encoded by the sulfonation sensitive and insensitive viral vectors. Usually, in a cell-based assay, one would want to test somewhere around 1-10 micromolar concentration of the test compound at the outset and then analyze the best inhibitors for their half maximal inhibitory concentration (IC₅₀) and for downstream structure activity relationship (SAR) analysis, etc.

The screen could be easily modified to allow for high-throughput screening of either siRNAs or overexpression clones. One would simply transfect the 293 cells with either the siRNA or expression vector and then infect, preferably the next day, with the two viruses. Essentially, transfection would replace chemical treatment.

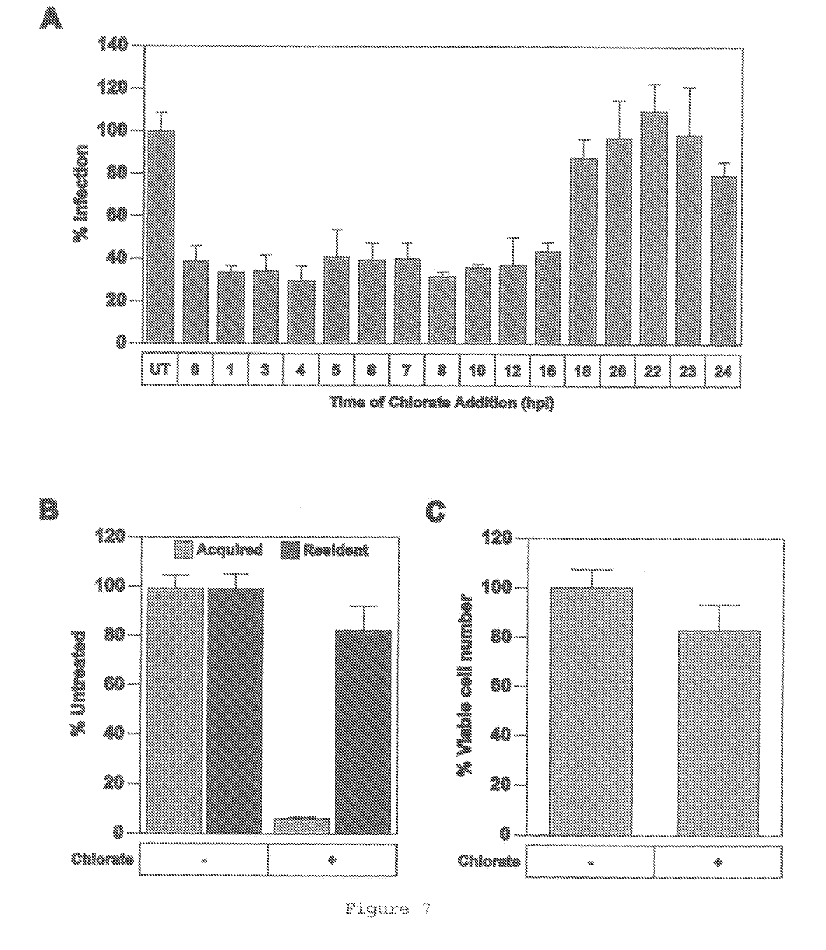

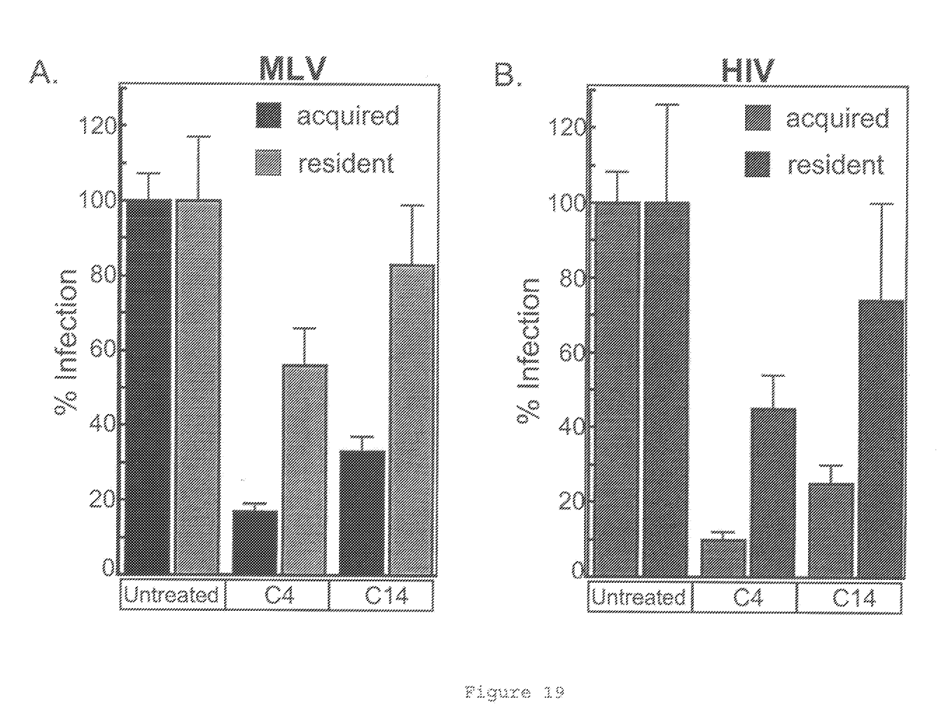

Viruses, test compound and cells may be incubated together, preferably for 48 hours, and the cells will be harvested for reporter assays. Based on our experience with two known inhibitors of this pathway, chlorate and guaiacol, we know chemicals can be added at the start of infection and up to 18 hours post infection (FIG. 7). This is demonstrated in Example 1. At later time points, after integration is complete, the virus becomes insensitive to sulfonation inhibitor treatment. This feature has been used as a counterscreen in the most recent assays to confirm that the new inhibitors block retroviral infection at the same step as other sulfonation inhibitors (FIG. 19). Several of our compounds inhibit expression from newly acquired virus but have little activity on previously established, integrated provirus. Therefore, in one version of the invention, the test chemical compound is added before the viral vectors. In other versions, the test agent is added at the same time or after the viral vectors are introduced to the cell. In another embodiment of the invention it is possible to pre-treat cells with a compound, but the compound should not be removed until after integration is complete.

A suitable reporter could be an enzyme (e.g. luciferase or β-galactosidase, etc.) or a fluorescent protein (e.g. GFP). The potential of each test chemical or compound as a sulfonation inhibitor will be tested by infecting cells with either a sulfonation sensitive HIV vector encoding luciferase or a sulfonation insensitive HIV vector encoding β-galactosidase in the presence of the compound. The efficacy of each compound as a sulfonation inhibitor will be scored by calculating the ratio of the reporter gene expression from the sensitive and insensitive viruses. In the specific assay described above, sulfonation inhibitors are predicted to have low luciferase but high β-galactosidase numbers (thus resulting in a low ratio relative to untreated controls). Untreated cells, as well as chlorate and guaiacol treated cells, will be included as controls on each plate. Appropriate statistical analysis will be applied to ensure the quality and reproducibility of the data set.

We envision multiple methods of measuring a test chemical or compound's inhibition of the sulfotransferase pathway. In one embodiment the cells can be stained post infection using X-gal or any compound suitable for indicating the existence of reporters. In a preferred embodiment, the cells are assayed for reporter enzyme activity or viable cell number using a chemiluminescent assay.

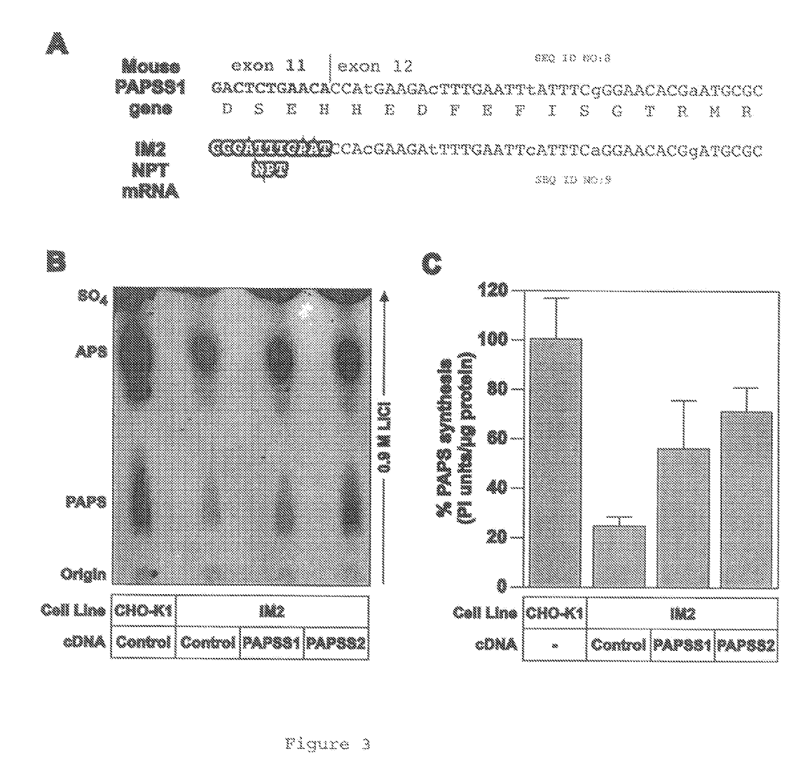

An in vivo screen, as disclosed below, is probably a best option because the method bypasses viability, bio-availability, and virus specificity issues. However, one could screen directly for inhibitors of PAPSS1 and sulfotransferases in general. One could screen for inhibitors of PAPSS enzymes using an in vitro assay such as that shown in FIGS. 3B and 3C. This is not high throughput screening, but it could be feasible for testing small libraries. Sulfotransferase inhibitors could be screened by assessing their effects on sulfation of specific targets, e.g. TPST1 and TPST2 sulfate tyrosines on cell surface proteins and one could look for the specific loss of this modification.

The most preferable assays for these proteins would typically be in vitro assays using purified protein expressed and purified from bacteria. For assaying the effect of a compound on PAPSS1 or PAPSS2, the purified enzymes would be mixed with ATP and ^{[35]}S labeled inorganic sulfate, and the conversion of ATP to PAPS could be followed by thin layer chromatography as in FIG. 3. PAPSS1 purification and enzymatic assays are described in Venkatachalam et al, JBC 273:30, 19311-19320, Jul. 24, 1988. Alternatively, a more rapid and quantitative method would be to modify commercially available assays that are used to measure kinase activity by loss of ATP (Promega).

The simplest assay for measuring sulfotransferase activity is to follow the transfer of ^{[35}]SO₄ from PAPS to a substrate. However, for a high-throughput assay to measure the effect of the compounds on sulfotransferases, we would modify the procedure of Burkart and Wong (Anal. Biochem. 274, 131-137, 1999). This is a coupled enzyme assay in which any sulfotransferase can be incubated in the presence of a target substrate PAPS and an inhibitor. The sulfotransferase produces sulfonated substrate and PAP. PAP can then be converted back into PAPS by the action of the enzyme β-AST-IV on its substrate p-nitrophenyl sulfate. Removal of sulfate from p-nitrophenyl sulfate generates PAPS and the p-nitrophenylate. Production of p-nitrophenylate can be followed by its absorbance at 410 nm and is quantitative, although indirect of sulfotransferase activity.

Alternatively, a modification of the dot blot assay designed by Verdugo and Bertozzi (Anal. Biochem. 307, 330-336, 2002), (Hemmerich et al., DDT 9:22, 967-975, November 2004) could be used. A radioactive or fluorescently tagged target is incubated with sulfotransferase and PAPS, the addition of a negative charge makes the substrate bind to a positively charged filter which is then washed and assayed for the amount of fluorescent or radioactive substrate bound.

F. Secondary Tests

Once potential candidates have been identified, the test compounds will typically be retested, preferably with MLV (another sulfotransferase sensitive virus) and ASLV (a sulfonation insensitive virus), to verify the effect. However, any vector having LTRs that are sensitive to sulfonation pathway inhibition can be used in place of MLV, and any vector having LTRs that are not sensitive to inhibition of this pathway can be used in place of ASLV. The effects of the compounds on the different stages of the viral life cycle could be further verified by well-established PCR-based methods. The effects of the compounds on cell viability can also be directly measured.

Neither the MLV nor the ASLV vectors are commercially available, but both have been in circulation for years. The ASLV vector is from Federspiel et al. (Proc. Nat'l Acad. Sci. USA 91:11241-11245, 1994). The MLV vector is from R. C. Mulligan et al. (Proc. Nat'l Acad. Sci. USA, 93: 11400-11406, 1996). Suitable MLV vectors are commercially available (some have been discontinued) from multiple sources including pLib from Clontech.

We envision that potential targets of the screen fall into three distinct categories: transporters; PAPSS inhibitors; and sulfotransferase inhibitors. There are at least two types of transporters involved in sulfonation—sulfate transporters that bring inorganic sulfate into the cell and transporters that move PAPS from the cytoplasm into the ER/golgi. Inhibitors of sulfate transporters could take the form of general anion transport inhibitors such as 4,4′-diisothiocyanostilbene-2,2′-disulfonic acid (DIDS) (Elgavish, et al. (1991) Am. Physiol. Soc'y, C916-C926).

No known in vivo inhibitors of transport of PAPS into the Golgi are known. Inhibition of PAPSS is the best studied of the sulfonation pathway proteins. The inhibition can occur competitively with sulfate analogs such as chlorate, which inhibits PAPSS activity at 100 mM in media that contains sulfate or at less than 1 mM in media lacking sulfate. Similar sulfate analogs have been analyzed for their effect on sulfonation of secreted cellular proteins (Hortin et al., Biochem. Biophys. Res. Comm., 150(1):342-348, 1988). Alternative inhibitors may interfere with ATP binding or formation of the reaction intermediate APS.

Sulfotransferase inhibitors would be predicted to either interfere with PAPS binding or act as substrate analogs. Guaiacol and trichlosan interfere with aryl sulfotransferases in this manner in mM and μM quantities, respectively (Hortin et al., Biochem. Biophys. Res. Comm., 150(1):342-348, 1988; Wang et al., Drug Metabolism Disposition, 32(10):1162-1169, 2004).

G. Treatment Methods

In another embodiment, the present invention is a method of treating an HIV infected individual with an inhibitor of the present invention. In a preferred embodiment, the treatment would be as a prophylactic agent, such as in spermicides. In one embodiment of the invention, high levels of the inhibitor compound could reduce infection at the earliest points. A likely use of the inhibitor would be as part of oral combinatorial therapy such as is currently in use with combinations of reverse transcriptase (RT), integrase (INT), and protease inhibitor cocktails.

The idea is to reduce viral titers in patients by reducing the expression of proviruses that successfully infected. In a preferred embodiment, this would be used as a combinatorial therapy similar to current protease inhibitors. Current protease inhibitors are used in combination with RT and INT inhibitors, which prevent the establishment of infection. In those cells infected because the virus escaped the RT and INT inhibitors (through chance or mutation), the protease inhibitors block the maturation of viruses coming out of these cells resulting in a significantly reduced amount of mature infectious virus. These lower titers reduce the severity of the infection in the host and lower the chance of transmission. Similarly, sulfonation inhibitors would be added to a cocktail of RT, INT, and protease inhibitors. The cells that are successfully infected would produce much less viral RNA and therefore much lower viral titers. This again would reduce the severity of disease and transmission.

Combination therapy works because although viruses rapidly mutate under selective pressure, it is difficult to obtain multiple drug resistance mutations that target different viral genes and still replicate well. Furthermore, since this is a cellular target, it may be more difficult for the virus to mutate and avoid.

In one embodiment, the present invention is a treatment for HIV. In order to develop a treatment dose, one would first optimize the system using dose response curves in tissue culture. Next, one would test WT virus in tissue culture. Then, one could use animal models using simian immunodeficiency virus (SIV) infected rhesus macaques or initially testing the efficacy of blocking MLV infection in mice.

In another embodiment, the present invention is used to prevent HIV in the form of a microbicide applied prior to sexual activity.

### Example 1

Materials and Methods

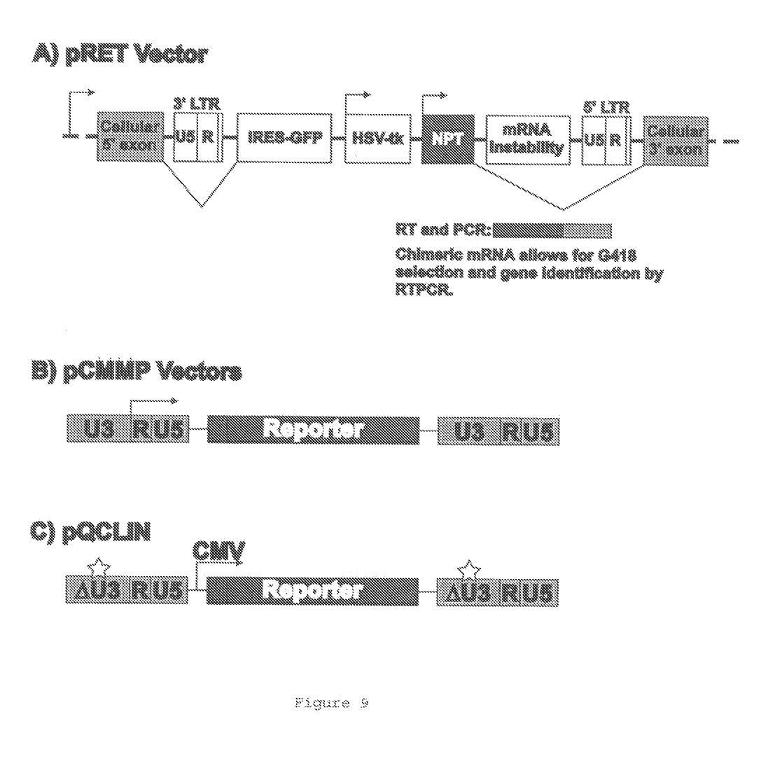

Plasmids and viral vectors: A schematic of the proviral forms of the MLV constructs used in this paper is provided in FIG. 9. The viral genome plasmids pMMP-nls-LacZ, pCMMP-eGFP and pCMMP-IRES-GFP, pCMMP-CD4-eGFP, pHIV-TVA800-hcRED, pRET and the ASLV-A genome plasmid RCASBP(A)-AP have been previously described (Boerger et al, Proc. Nat's Acad. Sci. USA 96:9867-9872, 1999; Melikyan et al., J. Virology 78:3753-3762, 2004; Federspiel et al.; Proc. Nat'l Acad. Sci. USA 91:11241-11245, 1994). The MLV vectors pLEGFP (Clontech, Palo Alto, Calif.) and pQCLIN (Clontech, Palo Alto, Calif.) as well as the HIV-1 self inactivating (SIN) pLenti6/V5-GW/lacZ (Invitrogen, Carlsbad, Calif.) were obtained commercially. The HIV-1 vector pNL4-3.Luc.R-E- (Connor et al., Virology 206:935-944, 1995) was obtained from the NIH AIDS Research and Reference Reagent Program, Division of AIDS, NIAID, NIH (deposited by Dr. Nathaniel Landau).

To construct the MLV vector pCMMP-CD4 (expressing human CD4 from the viral LTR), the previously described pCMMP-CD4-eGFP vector (Bruce et al., J. Virology 79:12969-12978, 2005) was digested with PmII and HpaI to remove the IRES-eGFP cassette and then the plasmid was re-ligated. The MLV vector pCMMP-HcRED (encoding the red fluorescent protein HcRED from the viral LTR) was generated by removing the multiple cloning site and IRES from pCMMP-IRES-GFP by AgeI/HpaI digestion and inserting an AgeI/StuI fragment containing the HcRED coding sequence from pHcRED1 (Clontech, Palo Alto, Calif.). The MLV vector pCMMP-SEAP-IRES-GFP (encoding SEAP and GFP) was generated by inserting the SEAP gene from pSEAP-control (Clontech, Palo Alto, Calif.) upstream of the IRES in pCMMP-IRES-GFP. HIV-1 vectors for stable expression of PAPS synthases or control cDNAs were generated by PCR amplification of coding sequence (PAPSS1: IMAGE#3869484, PAPSS2: IMAGE#2988345, control cDNA ZNF639: IMAGE#4794621) from commercially available cDNAs (Open Biosystems) and cloning into MluI/EcoRV digested pLenti6/V5-GW/lacZ.

Cell culture and virus production: Chinese hamster ovary cells (CHO-K1, ATCC CCL-61) were cultured in F-12 media supplemented with 10% bovine calf serum (BCS) (Invitrogen, Carlsbad, Calif.). Human embryonic Kidney 293T cells (ATCC CRL-11268) were cultured in Dulbecco's Modified Eagle's Medium (DMEM) supplemented with 10% fetal calf serum (FCS) (Hyclone, Logan, Utah). Chicken DF-1 cells (ATCC CRL-12203) were cultured in DMEM supplemented with 10% FCS. Jurkat cells (ATCC TIB-152) were cultured in RPMI-1640 supplemented with 10% FCS.

CHO cells expressing the receptor for ASLV (TVA-800) were generated as previously described (Bruce et al., J. Virology 79:12969-12978, 2005) by infection with HIV-1-TVA800-hcRED[VSV-G] at an approximate moi of 0.5 hcRED transducing units for 2 hours. Cells infected with this virus express TVA800, HcRED, and the blasticidin S deaminase (BSD) gene. Infected cells were selected for two weeks in the presence of 3 Mg/ml blasticidin (Invitrogen, Carlsbad, Calif.). Cells expressing either PAPSS1, PAPSS2 or the control cDNA, ZNF639, were generated by infecting CHO-K1 and IM2 cells with VSV-G pseudotyped HIV-1 vectors encoding the appropriate open reading frame (ORF) (see above) at an approximate moi of 0.5 blasticidin transducing units for 2 hours. Infected cells were selected for two weeks in 3 μg/ml blasticidin.

MLV VSV-G and EnvA pseudotyped viruses were generated by calcium phosphate transfection of 293T cells as previously described (Boerger et al., Proc. Nat'l Acad. Sci. USA 96:9867-9872, 1999; Landau et al., J. Virology 66:5110-5113, 1992; Bruce et al., J. Virology 79:12969-12978, 2005). VSV-G pseudotyped HIV-1 vector was produced by a similar procedure except the genome plasmid used was pNL4-3.Luc.R-E-. The VSV-G pseudotyped self-inactivating HIV-1 vector was made using the Virapower kit (Invitrogen, Carlsbad, Calif.) following manufacturers instructions. DF-1 cells were transfected using the calcium phosphate method with the subgroup A-specific ASLV-A vector, RCASBP (A)-AP, encoding alkaline phosphatase (Federspiel et al., Proc. Nat'l Acad. Sci. USA 91:11241-11245, 1994). Media from transfected cells was collected 2 days post transfection to 7 days post transfection and filtered through a 0.45 μm bottle top filter. Virus was stored at 4° C. through the collection period, combined and then frozen at −80° C. for long-term storage. Virus for use in Quantitative PCR amplification studies was treated with DNaseI (Roche Applied Science, Indianapolis, Ind.) to remove contaminating plasmid DNA from the virus preps. DNaseI was added as a powder to a final concentration of 1 μg/ml when the virus containing supernatants were collected. The supernatants were incubated 1 hr at room temperature before filtration.

The titer of VSV-G and envA vector stocks were determined by assaying for transduction of a marker gene following infection of either WT CHO-K1 cells or WT CHO-K1 cells that had been engineered to express TVA800 (Bruce et al., J. Virology 79:12969-12978, 2005). For viruses that lack a cell-based reporter gene assay, immunoblot analysis of viral capsid protein (CA) levels (α-p24 for HIV or α-p36 for MLV) in the extracellular supernatants of producer cells was used to equalize the amounts of input virus as compared to those associated with viral vectors that contain reporter genes (Lenti6/V5-GW/lacZ[VSV-G] for HIV and MMP-nls-LacZ[VSV-G] for MLV).

Retroviral Insertional mutagenesis and isolation of an MLV-resistant clone: CHO-K1 cells (1×10⁸) were mutagenized by infection with VSV pseudotyped pRET at an approximate moi of 0.01 GFP transducing units. Cells were selected in 900 μg/ml G418 for two weeks. A pool of 2×10⁷ insertionally mutagenized CHO-K1 cells were challenged with CMMP-CD4 [VSV-G] at an approximate moi of 1 CD4 transducing units for two hours at 37° C. in the presence of 4 μg/ml polybrene. Unbound viruses were then removed and fresh medium was added. At 48 hours post infection (hpi) the cells were removed from the plate with phosphate buffered saline (PBS) containing 5 mM ethylenediaminetetraacetic acid (EDTA). Cells were pelleted (200×g, 5 min) and resuspended in 500 μl PBS containing 2 mM EDTA and 2% bovine serum albumin (BSA) (Sigma-Aldrich, Inc., St. Louis, Mo.). The cells were incubated with anti-human CD4 iron-conjugated antibody (Miltenyi Biotec Inc., Auburn, Calif.) at 20 μg/10⁷ cells for 15 minutes at 4° C.

Large cell (LC) columns (Miltenyi Biotec Inc., Auburn, Calif.) were applied to a magnetic field and washed with 2 ml PBS containing 2 mM EDTA and 2% BSA. Cells were filtered through a 30 μm mesh (Miltenyi Biotec Inc, Auburn, Calif.) and applied to the LC column. Cells were washed twice with 2 ml PBS containing 2 mM EDTA and 2% BSA. Column flow through and washes were collected and the cells were pelleted, resuspended in medium, and replated. Cells were allowed to recover for at least 16 hours before the next viral challenge. When necessary, the cells were expanded between each round of virus challenge to a minimum of 5×10⁵ cells per sort. The challenge and selections were repeated five times. The population was challenged a final time with CMMP-HcRED[VSV-G] and the HcRed negative cells were single cell cloned after high speed FACS (University of Wisconsin Comprehensive Cancer Center).

Single cell clones from the sorted insertional mutant pools were grown for 14 days post sorting, trypsinized and then plated onto duplicate assay plates. The assay plates were incubated for 2 hours with pMMP-nls-LacZ [VSV-G] at an approximate moi of 1 LacZ transducing unit (LTU) in the presence of 4 μg/ml polybrene. Unbound virus was then removed and fresh medium was added. At 48 hpi, one plate was assayed for (3-galactosidase activity using the Galacto-Star chemiluminescent kit (Applied Biosystems, Foster City, Calif.) according to the manufacturers instructions and the other plate was assayed for cell number and cell viability using CellTiter-Glo reagent (Promega, Madison, Wis.) following the manufacturers instructions to control for variations in cell number among the clones.

Chemiluminescent assay of viral infection: Quantitative chemiluminescent infection assays were performed as previously described (Bruce et al., J. Virology 79:12969-12978, 2005). Briefly, 8 wells of a 96 well plate were seeded at 1×10⁴ cells/well for each cell line tested. The cells were incubated for 2 hours with an approximate moi of 1 transducing unit (based on marker gene expression for β-galactosidase and alkaline phosphatase, or CA equivalents for luciferase, as described above), in the presence of 4 μg/ml polybrene. Unbound virions were removed and fresh medium was added. At 48 hpi, four wells were assayed for β-galactosidase activity using the Galacto-star Kit (Applied Biosystems, Foster City, Calif.), for alkaline phosphatase activity using the Phospha-Light Kit (Applied Biosystems, Foster City, Calif.) or for luciferase activity using the Britelite (PerkinElmer, Boston, Mass.) according to the manufacturer's instructions. The other four wells were assayed for cell number and cell viability using CellTiter-Glo reagent (Promega, Madison, Wis.) as described above. The results obtained were normalized for relative cell number.

To determine the absolute fold-resistance to viral infection, X-Gal staining was performed on cells that were infected with serial dilutions of viruses. For these experiments, cells were seeded at 1×10⁴ cells/well in triplicate rows for each cell line tested. The cells were then infected for 2 hours with ten-fold serial dilutions of MMP-nls-LacZ [VSV-G] in the presence of 4 μg/ml polybrene as described before and the cells were subsequently stained with X-gal as previously described (Adkins et al., J. Virology 75:3520-3526, 2001]. The blue cells contained in wells that had between 20 and 200 β-galactosidase positive cells were counted to give an accurate measure of the viral titer.

PAPS assays: PAPS assays were performed as previously described (Venkatachalam et al., J. Biol. Chem. 273:19311-19320, 1998]. Briefly, cells were lysed by three freeze thaw cycles in PAPS lysis buffer [20 mM tris(hydroxymethyl)aminomethane (Tris) pH8, 20% sucrose, 1 mM EDTA, 1 mM (DTT)] in the presence of 1× protease inhibitor cocktail (RPI, Mt. Prospect, Ill.). Cell lysate (1 μl) was mixed with 5 mM ATP and 10 μCi ^{[35]}S labeled sulfate in reaction buffer [50 mM Tris pH8, 25 mM MgCl₂, 0.9 M EDTA, 13.5 mM DTT) and incubated for 30 minutes at room temperature. Thin layer chromatography (TLC) was used to separate PAPS, APS and SO₄ on PEI cellulose TLC plate (EMD Chemicals, Gibbstown, N.J.) in 0.9 M LiCl. TLC plates were dried, exposed to phosphoimager plates, and quantified using the Imagequant software volume method. Mobility positions were confirmed with commercial PAPS preparations (PerkinElmer, Waltham Mass., Cat# NEG010100UC). Each sample was normalized for μg of total protein in the lysate determined by Bradford assay using the Quick Start Bradford Dye reagent (Bio-rad, Hercules, Calif.).

Real time quantitative PCR: To measure the amounts of reverse transcription intermediates in infected cells, cells were seeded in triplicate wells at 5×10⁵/well in a 6 well plate and then infected at 4° C. on a rocking platform at an moi of 1 GFP transducing unit (GTU) for 2 hours with an MLV vector (pLEGFP; Clontech, Palo Alto, Calif.) pseudotyped with VSV-G that was treated with DNaseI as described above. Virus derived from pLEGFP was used for these assays because the 3′ viral LTR varied enough from pCMMP so real-time PCR primers could be designed that specifically recognized the pLEGFP derived test virus but not the pCMMP derived screen virus.

DNA was harvested from infected cells 24 hpi using the DNeasy Kit (Qiagen, Valencia, Calif.). For the nuclear fractionation studies nuclei were harvested from infected cells 24 hpi using the Nuclei EZ Prep Kit (Sigma-Aldrich, Inc., St. Louis, Mo.) following the manufacturers instructions and DNA was isolated from nuclei as described above. To measure integrated proviral DNA copy number, cells were seeded and infected as described above and then passaged for 18 days. DNA was then harvested from 1×10⁶ cells as described above. DNA concentration was calculated by measuring the A260 on a SPECTRAmax Plus 96 well UV spectrophotometer (Molecular Devices, Sunnyvale, Calif.). Quantitative, real time PCR (QPCR) analysis was performed on an ABI 9600 (Applied Biosystems, Foster City, Calif.) using the standard cycling conditions of 50° C. 10 min, 40 cycles of 95° C. 30 s, 60° C. 2 minutes. DNA (10 p1125 μl reaction) was amplified in TaqMan Universal PCR Mastermix (Applied Biosystems, Foster City, Calif.) with 1 μM each primer and 0.1 μM 5′, 6-FAM, 3′TAMRA labeled probe. Each primer probe set was tested on each cell line in a minimum of 3 independent experiments.

The number of molecules in each reaction was determined by comparison to standard curves generated from amplification of plasmid DNA containing the target sequence. The primers used are specific for the U3-U5 region of the LEGFP vector and are shown along with the viral LTR feature and the by position recognized in pLEGFP are:

SEQ ID NO: 1)
OJWB39
(5′-CAGTTC GCTTCTCGCTTCTGTTC-3′,
[U3, bp 523-535],
SEQ ID NO: 2)
OJWB47
(5′-GTCGTGGGTAGT CAAT CACTC AG-3′,
[R and U5, bp 697-719]
and
SEQ ID NO: 3)
OJWB38
(5′-6-FAM-ATCCGA ATCGTGGTCTCGCTGTTC-TAMRA-3′,
[R, bp 657-680].

RNase Protection Assays: Templates for RNA probes to MLV were generated by PCR amplification using 1 μg total DNA from CHO-K1 cells infected with CMMP-GFP[VSV-G] along with the oligonucleotide primers OJWB7 (5′-GAACAGATGGTCCCCAGATGC-3′, SEQ ID NO:4) and OJWB8 (5′-CGGTGGAACCTCCAAATGAA-3′, SEQ ID NO:5). ExTaq polymerase (Takara, Madison Wis.) was used with cycling conditions of [95° C. 5 min, 30 cycles of 95° C. 30 S, 50° C. 30 S 72° C. 1 min]. This resulting LTR fragment was cloned into pGem T-easy (Promega, Madison, Wis.) and spanned 192 bp upstream of the transcription start (+1, the start of R) to 139 bp downstream of +1, which results in a 140 bp protected fragment in the RNase protection assays.

The template for RNA probes to hamster actin RNA were generated by reverse transcription of the hamster p-actin cDNA cloned by reverse transcription PCR amplification of 1 μg total RNA isolated from CHO-K1 cells with OJWB313 (5′-TCACCCACACTGTGCCC ATCTATGA-3′, SEQ ID NO:6) and OJWB314 (5′-CAACGGAACCGCTCATTGCCAATGG-3′, SEQ ID NO:7) and MasterAmp tTh polymerase (Epicenter, Madison Wis.) using cycling conditions of [60° C. 5 min, 30 cycles of 95° C. 30 S, 50° C. 30 S 72° C. 1 min]. The resulting PCR amplified product was cloned into pGem T-easy (Promega, Madison, Wis.) and generates a 294 bp protected fragment in RNase protection assays. Anti-sense RNA probes were generated by digesting the plasmids with SpeI and performing in vitro transcription reaction using the Riboscribe Kit (Epicenter, Madison Wis.) with T7 polymerase and 50 μCi α-^{[32]}P-UTP.

To measure the amounts of transcription from integrated proviruses in infected cells, cells were seeded in triplicate wells at 5×10⁵/well in a 6 well plate and then infected at 4° C. on a rocking platform at an moi of 1 GTU for 2 hours with an MLV vector (pCMMP-GFP) pseudotyped with VSV-G. RNA was isolated from cells 24 hpi using the RNeasy kit (Qiagen, Valencia, Calif.) following manufacturers instructions. RNase protection assays were performed by mixing 2 μg (viral transcripts) or 0.5 μg (1′-actin) of total RNA with 5×10⁴ cpm probe, hybridizations and digestions were done using the RPA III kit (Ambion, Austin Tex.). Protected fragments were separated on a 6% PAGE-Urea gel, dried and exposed to a phosphoimager plate. Phosphorimage units were measured using the Imagequant software volume method.

Results

Isolation of the IM2 cell line resistant to infection by a MLV vector: Chinese hamster ovary (CHO-K1) cells were used for insertional mutagenesis by a retroviral vector since these cells are functionally hypodiploid at numerous loci (Gupta et al. Cell 14:1007-1013, 1978) and therefore insertion of the viral vector into a single allele of a given cellular gene can be sufficient to produce a genetically-null phenotype. The insertional mutagenesis was performed with the murine leukemia virus (MLV)-based vector pRET, which encodes green fluorescent protein (GFP), as well as a neomycin phosphotransferase (NPT) mRNA that contains an instability element downstream of a canonical splice donor site (Ishida et al., Nucleic Acids Res. 27:e35, 1999). Integration of pRET upstream of a cellular exon gives rise to a NPT mRNA transcript in which the instability element is removed by mRNA splicing, thereby conferring G418 resistance on the mutagenized cells (FIG. 9a).

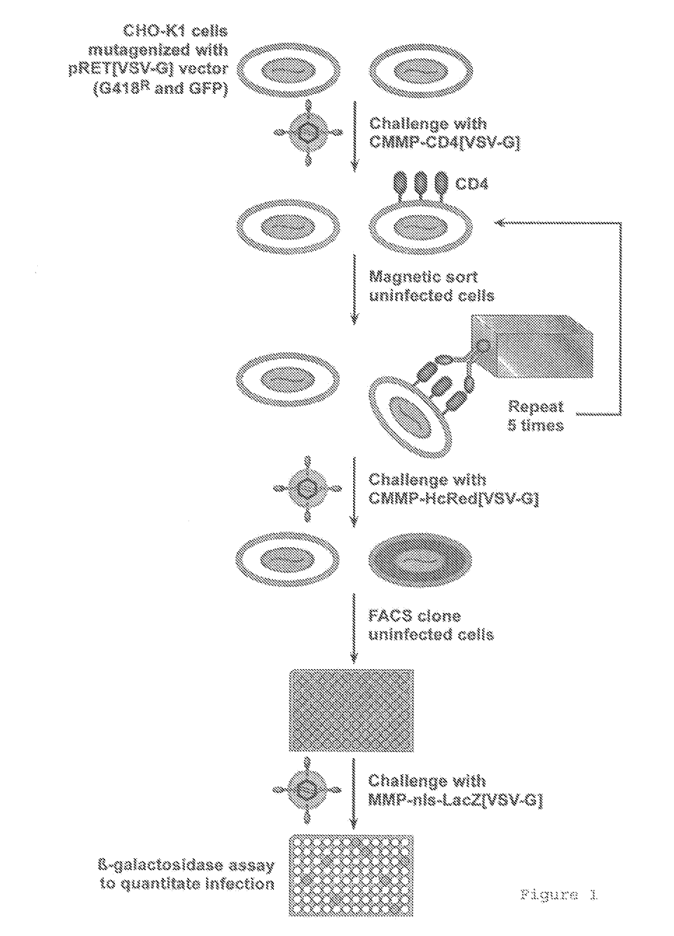

Approximately 1×10⁶ colonies of G418-resistant cells were generated by challenging CHO-K1 (1×10⁸) cells with VSV-G pseudotyped pRET at a moi of 0.01 (note: at this moi only a small fraction of these cells are “infected”) to ensure only one integration event per cell. Mutagenized cells were selected in a medium containing 900 μg/ml G418 for two weeks, after which the population was expanded and pooled. In order to identify cells in the population that were resistant to retroviral infection, a pool of 2×10⁷ insertionally mutagenized cells were subjected to five rounds of challenge with a second, replication-defective, VSV-G pseudotyped MLV vector which contains a human CD4 gene that is expressed from the viral promoter (FIG. 1). Infected cells that expressed human CD4 on their surface were removed from the population at each round by magnetic cell sorting (MACS) using an iron-conjugated CD4-specific antibody (FIG. 1).

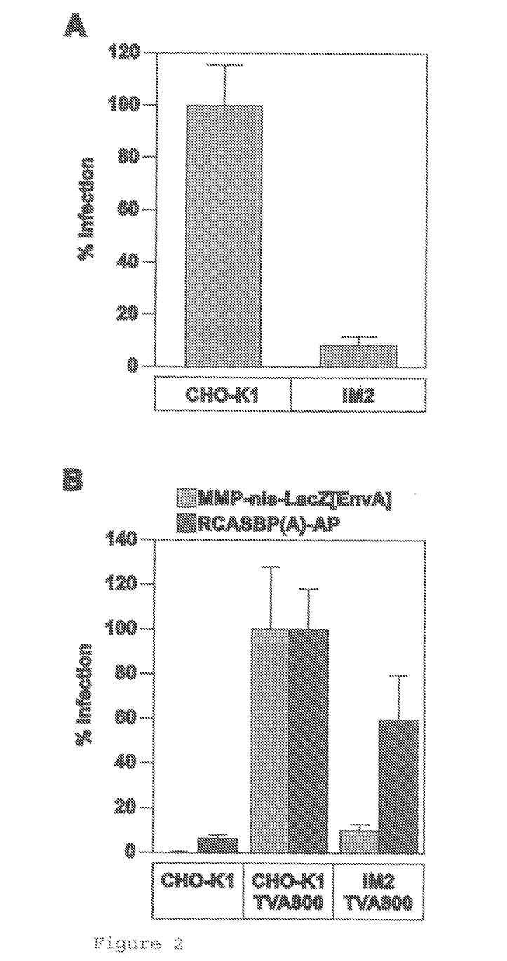

Each round of infection and sorting resulted in an approximate 3-fold enrichment of CD4-negative cells relative to the preceding round, with a total enrichment of 47-fold. The resultant cell population, which exhibited an overall 2.5-fold resistance to MLV infection, was then challenged a final time with another VSV-G pseudotyped MLV vector encoding the far-red fluorescent protein HcRed. A total of 264 single cell clones of HcRed-negative cells were then isolated by FACS (FIG. 1) and tested for their susceptibility to infection by a VSV-G pseudotyped MLV vector encoding β-galactosidase. One cell line, designated IM2, that was judged to be one of the most resistant (approximately 12-fold) to challenge by that viral vector, based upon viral reporter gene expression (FIG. 2A), is characterized in detail in this report.

To determine if the defect associated with the IM2 cell line is specific for the MLV vector, wild-type CHO-K1 cells and mutant IM2 cells were engineered to express TVA800, the cellular receptor for an avian retrovirus, subgroup A avian sarcoma and leukosis viruses (ASLV-A) (Bates et al., Cell 74:1043-1051, 1993; Young et al., J. Virology 67:1811-1816, 1993). The TVA800 expressing cells were then challenged with either the MLV vector encoding β-galactosidase vector pseudotyped with the ASLV-A envelope protein (EnvA) or instead with an ASLV-A vector that encodes heat-stable alkaline phosphatase (Federspiel et al., Proc. Nail Acad. Sci. USA 91:11241-11245, 1994).

Viral reporter gene expression following infection of IM2-TVA800 cells by the EnvA-pseudotyped MLV vector was 9.7-fold reduced as compared with CHO-K1-TVA800 cells (FIG. 2B). This effect mirrored that seen with VSV-G pseudotyped MLV vectors (e.g. FIG. 2A). Thus, the defect seen with IM2 cells is independent of the nature of the viral glycoprotein used to pseudotype the MLV vector. By contrast, the level of viral reporter gene expression following infection by the ASLV-A vector was comparable between IM2-TVA800 and CHO-K1 cells (FIG. 2B). Since both vectors utilized EnvA to mediate entry, these observations indicate that the defect associated with the IM2 cell line is specific for protein or RNA components of the MLV core.

The PAPSS1 gene is disrupted in IM2 cells: To identify which cellular gene was disrupted by the mutagenic pRET vector, total RNA was isolated from IM2 cells and reverse transcription PCR amplification was performed using primers anchored on the virally encoded NPT gene and the poly (A) tail. DNA sequence analysis of the PCR amplification products and a comparison with the sequenced mouse genome revealed that the pRET provirus had integrated upstream of exon 12 of the 5′ phospho-adenosine, 3′ phosphosulfate synthase 1 gene (PAPSS1) (FIG. 3A). The full sequence of hamster PAPSS1 gene and its corresponding mRNA product have not yet been reported. However, comparison with the cognate mouse gene indicates that, in IM2 cells, the pRET-encoded NPT open reading frame is fused by mRNA splicing to the third base of the codon encoding amino acid residue 579 of PAPSS1 (FIG. 3A).

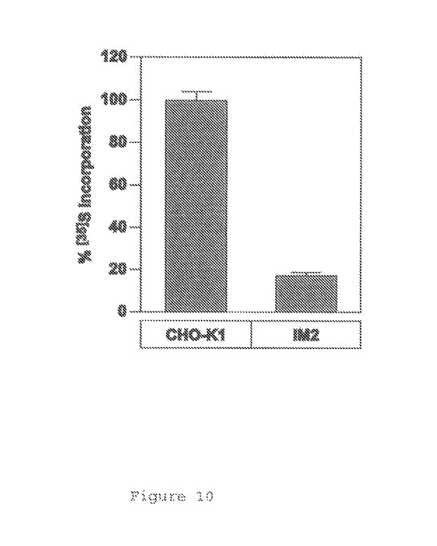

PAPSS1 and the highly related PAPSS2 enzyme catalyze the formation of the high energy sulfate donor 3′ phospho-adenosine, 5′ phosphosulfate (PAPS) (Venkatachalam et al., J. Biol. Chem. 273:19311-19320, 1998; Fuda et al., Biochem. J. 365:497-504, 2002; Girard et al., FASEB J. 12:603-612, 1998) used for all sulfonation reactions in the cell. Consistent with the prediction that IM2 cells have less PAPS available for sulfonation reactions, IM2 cells incorporated 17% less ^{[35]}SO₄ into macromolecules than CHO-K1 cells in bulk labeling experiments (FIG. 10). However, the readout of these experiments is several steps downstream of PAPS synthase and represents the summation of multiple enzyme/substrate interactions.

To directly determine if IM2 cells were deficient in PAPS synthase activity, an in vitro PAPS assay was performed. ATP and ^{[35]}SO₄ were mixed with cell lysates prepared from CHO-K1 cells, IM2 cells, or IM2 cells engineered to express human cDNA clones of either PAPSS1 or PAPSS2. The reaction products were separated on PEI cellulose TLC plates in 0.9M LiCl. (FIG. 3B) Inorganic sulfate exhibits the greatest mobility, followed by the reaction intermediate adenosine phosphosulfate (APS), with PAPS being retained closest to the origin (Fuda et al., Biochem. J. 365:497-504, 2002). Mobility positions were confirmed with commercial PAPS preparations (data not shown). TLC plates were exposed to phosphoimager plates and the levels of PAPS synthesized were measured. These studies demonstrated that IM2 cells have five-fold lower levels of PAPSS activity per μg of protein than do the parental CHO-K1 cells (FIGS. 3B and 3C). PAPS synthase activity in IM2 cells was significantly increased by stable expression of either human PAPSS1 or PAPSS2 cDNA clones (FIGS. 3B and 3C) although not to full WT levels. These data indicate that the pRET vector disrupted the function of the PAPSS1 gene in IM2 cells.

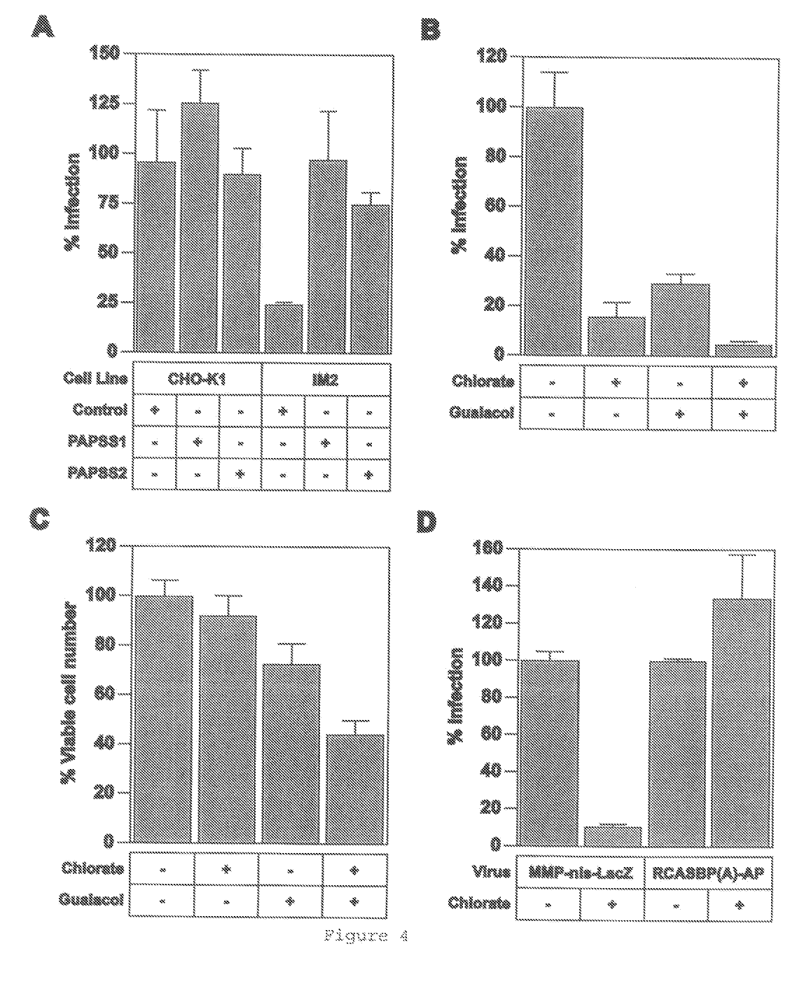

The cellular sulfonation pathway is required for MLV infection: To investigate whether the deficiency in PAPS synthase activity in IM2 cells was responsible for the block to MLV infection, CHO-K1 and IM2 cells engineered to express either human PAPSS1 or PAPSS2 were challenged with the VSV-G pseudotyped MLV vector encoding β-galactosidase and infected cells were enumerated by X-gal staining. Expression of either PAPSS enzyme complemented the MLV infection defect of the IM2 cell line (FIG. 4A). By contrast, a control cDNA, containing an ORF unrelated to sulfonation, did not rescue virus infectivity in these cells (FIG. 4A). These data confirm that the deficiency in PAPS synthase activity is responsible for the virus infection-resistant phenotype of IM2 cells.

To further investigate a role for the sulfonation pathway, CHO-K1 cells were treated with either chlorate, a substrate analog of sulfate and a competitive inhibitor of PAPS synthases (Girard et al., FASEB J. 12:603-612, 1998; Baeuerle et al., Biochem. Biophys. Res. Comm. 141:870-877, 1986; Hortin et al., Proc. Nat'l Acad. Sci. USA 86:1338-1342, 1989; Hortin et al., Biochem. Biophys. Res. Comm. 150:342-348, 1988), or with the sulfotransferase inhibitor guaiacol (Hortin et al., Proc. Nat'l Acad. Sci. USA 86:1338-1342, 1989; Hortin et al., Biochem. Biophys. Res. Comm. 150:342-348, 1988), prior to challenge with the MLV vector. As compared to untreated cells, chlorate-treated, guaiacol-treated, and chlorate/guaiacol dual-treated cells gave rise to approximately 6.7-fold, 3.4-fold, and 23-fold less blue cells, respectively (FIG. 4B).

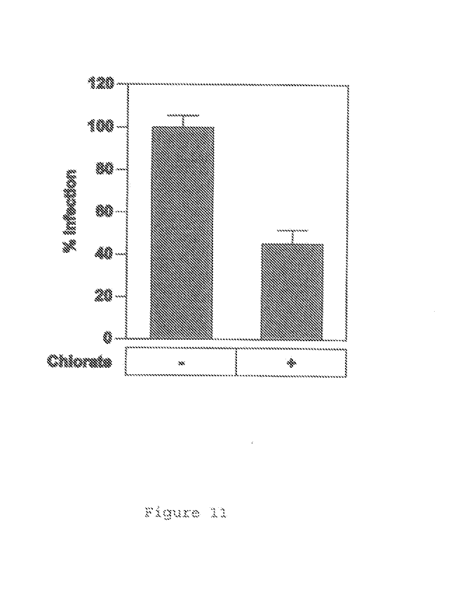

Only the dual inhibitor treatment led to a significant (2.3-fold) reduction in viable cell number (FIG. 4C), which was still considerably less than the effect on infection. Similarly, chicken DF1 cells treated with chlorate were approximately 9.3-fold less susceptible to infection by this viral vector as judged by reporter gene expression (FIG. 4D). However, this treatment did not influence infection of these avian cells by an ASLV-A vector. Treatment of IM2 cells with chlorate reduced MLV infection an additional 2-fold (FIG. 11), which is consistent with the observation that these cells contain some residual PAPS synthase activity (FIGS. 3B and 3C). These data further show a role for the cellular sulfonation pathway in infection by MLV, but not ASLV, vectors and indicate that the mechanism(s) responsible are shared between different host cell species.

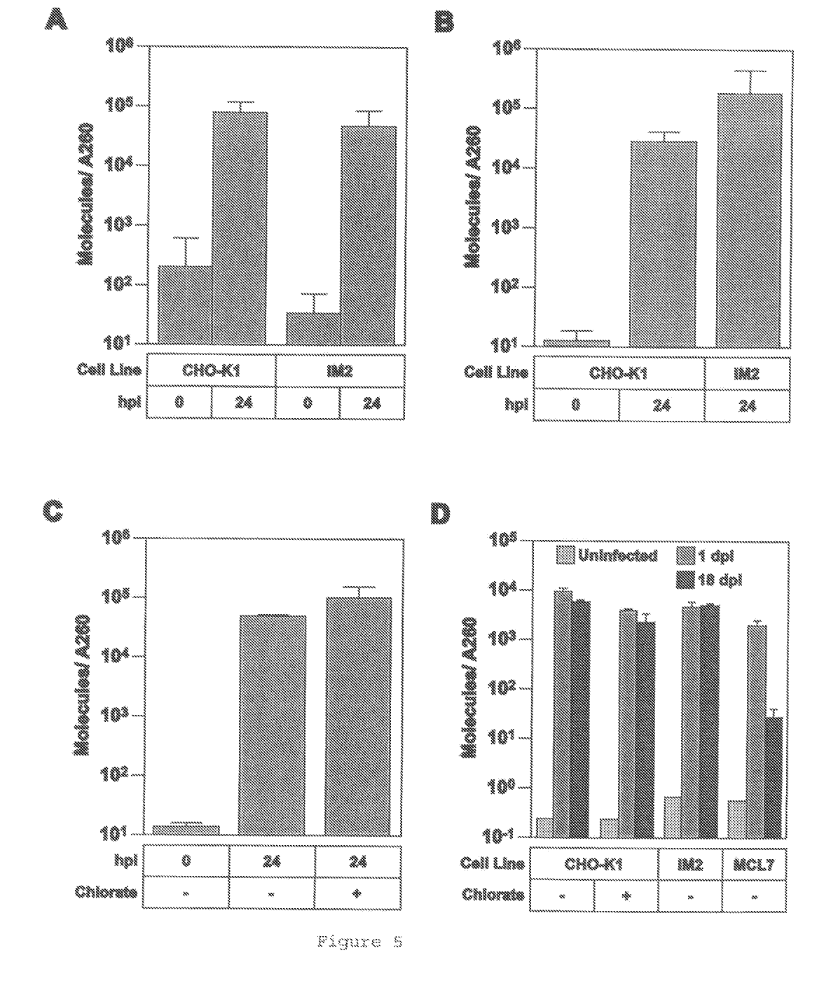

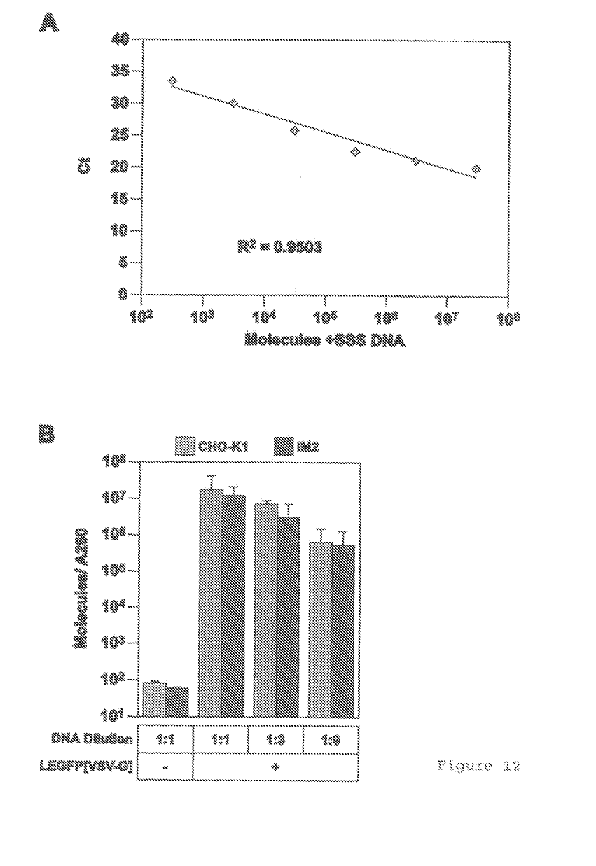

The sulfonation pathway does not influence viral reverse transcription or the level of proviral DNA: Real time PCR amplification was used to monitor the effect of the sulfonation pathway on the levels of reverse transcription products and integrated viral DNA. Cells were infected with an MLV vector (pLEGFP) and either total DNA or nuclear DNA was subsequently harvested. Since these cells potentially contain both the mutagenic pRET vector, and the pCMMP derived vector utilized in the screen, the primer/probe set was chosen to amplify the plus strand strong stop replication intermediate (Gotte et al., Arch. Biochem. Biophys. 365:199-210, 1999; Whitcomb et al., Ann. Rev. Cell Biol. 8:275-306, 1992) and annealed specifically to the unique 3 (U3) and unique 5 (U5) long terminal repeat (LTR) region of only the pLEGFP MLV vector (data not shown). This primer probe set exhibits an excellent dose response over 6 orders of magnitude (FIG. 12A) and a very low background, such that the signal from infected cells at 24 hpi is 400-fold higher than from cells where the virus is bound but not internalized (0 hpi, FIG. 5A) The difference between infected and uninfected cells is even greater (FIGS. 5D and 12B).

The levels of total and nuclear reverse transcription products were found to be the same in IM2 cells as in CHO-K1 cells (FIGS. 5A and 5B). Furthermore, treatment of CHO-K1 cells with chlorate had no effect on the accumulation of viral reverse transcription products, confirming that the sulfonation pathway does not influence viral DNA synthesis (FIG. 5C). Importantly, this is not due to saturation of the assay as dilution of input genomic DNA showed a proportionate decrease in both CHO-K1 and IM2 samples, even when a ten-fold higher multiplicity of infection was used (FIG. 12B). To investigate the possible role of this pathway in viral DNA integration, IM2 cells were infected with the same MLV vector, passaged for 18 days to allow loss of episomal forms of viral DNA (Zack et al., Cell 61: 213-222, 1992; Weller et al., J. Virology 33: 494-506, 1980), and the levels of total viral DNA were then measured.

IM2 cells and chlorate treated CHO-K1 cells contained nearly the same amounts of integrated viral DNA as untreated CHO-K1 cells (1.1 and 2.6-fold less, respectively) (FIG. 5D), which is insufficient to explain the approximately 10-fold decrease in infectivity (FIGS. 2A and 4B). By comparison at 18 days post-infection, nearly 400-fold lower levels of viral vector DNA were detected in MCL7 cells, a chemically mutagenized CHO-K1 cell line that exhibits a strong block to MLV DNA integration (Bruce et al., J. Virology 79:12969-12978, 2005) (FIG. 5D). These data demonstrate that the cellular sulfonation pathway does not influence either the levels of viral DNA that are synthesized in the target cell or that become integrated into the host cell genome.

The sulfonation pathway influences gene expression from the MLV LTR. Since the sulfonation pathway did not influence the level of integrated viral DNA, we next determined if it impacts subsequent provirus gene expression. In these studies, the level of MLV LTR-driven transcription from the MMP-nls-LacZ vector was compared to that from the internal CMV promoter contained in QCLIN, a commercially available, self-inactivating (SIN) MLV vector with promoter defective LTRs (Julius et at, Biotechniques. 28:702-708, 2000). The levels of β-galactosidase from the QCLIN vector were the same in infected IM2 and CHO-K1 cells (FIG. 6A), a result that supports our observation that the sulfonation pathway does not influence the overall level of viral DNA integration.

By striking contrast, MLV LTR-driven reporter gene expression following infection was reduced 5.6-fold in IM2 cells as compared with CHO-K1 cells (FIG. 6A). Consistently, a combination of chlorate and guaiacol treatment reduced β-galactosidase levels produced from the MMP-nls-lacZ vector by 7.3-fold, following infection of CHO-K1 cells, but this treatment did not influence gene expression from the SIN vector (FIG. 6B). These data suggest that the target of action for the cellular sulfonation pathway is contained within the MLV LTR.

To directly examine the influence of the sulfonation pathway upon MLV LTR-driven mRNA transcription, total RNA was isolated from CHO-K1 and IM2 cells that were infected with a VSV G-pseudotyped MLV vector encoding EGFP. RNase protection assays were performed with a probe that hybridizes to the primary viral mRNA transcript (hybridizing to the R-U5 region). IM2 cells accumulated 3.5-fold less primary transcript than CHO-K1 cells (FIGS. 6C and 6E). Similarly, the levels of viral-derived transcript were reduced in CHO-K1 cells treated with inhibitors of the cellular sulfonation pathway (Chlorate 11-fold, guaiacol 17-fold, and chlorate and guaiacol 40-fold (FIGS. 6D and 6E). All values were normalized to hamster β-actin levels, which varied less than 2-fold in all cases (FIGS. 6C and 6D). These data indicate that the sulfonation pathway influences a step that impacts the transcriptional competency of the provirus.

The sulfonation pathway acts at a step during provirus establishment: To determine the time point during infection when the cellular sulfonation pathway is involved, CHO-K1 cells were incubated with the VSV-G pseudotyped MLV vector encoding β-galactosidase at 4° C., and infection was then initiated by a temperature shift to 37° C. Chlorate was then added at various times post-infection and the effect of this treatment on the establishment of viral vector in these cells was then measured by quantitating β-galactosidase expression. Chlorate addition up to 16 hpi led to a reduction in subsequent viral reporter gene expression (FIG. 7A). However, addition of the inhibitor at time points 18 hpi, or later, had no effect (FIG. 7A). This timing coincides with maximal levels of viral DNA integration (Roe et al., EMBO. J. 12:2099-2108, 1993), suggesting that the cellular sulfonation pathway might influence a step during or shortly after provirus establishment.

To explore this possibility further we compared the effect of chlorate treatment on proviral gene expression from resident, versus newly acquired, proviruses. A CHO-K1 cell line was established that contains a resident MLV vector encoding secreted alkaline phosphatase (FIG. 7B). These cells were then challenged with the MLV vector encoding β-galactosidase in the presence of chlorate to generate newly acquired MLV proviruses under conditions where the sulfonation pathway was inhibited. These experiments showed that the chlorate treatment affected gene expression from the newly acquired, but not the resident proviruses (FIG. 7B). In an independent experiment, chlorate treatment was shown not to influence β-galactosidase expression from a resident MLV vector (data not shown), confirming that the effect seen was not reporter gene-specific. Taken together with the timing of the sulfonation requirement during infection (FIG. 7A), these results strongly imply that this cellular pathway influences MLV replication at a step during provirus establishment, one that impacts subsequent viral gene expression.

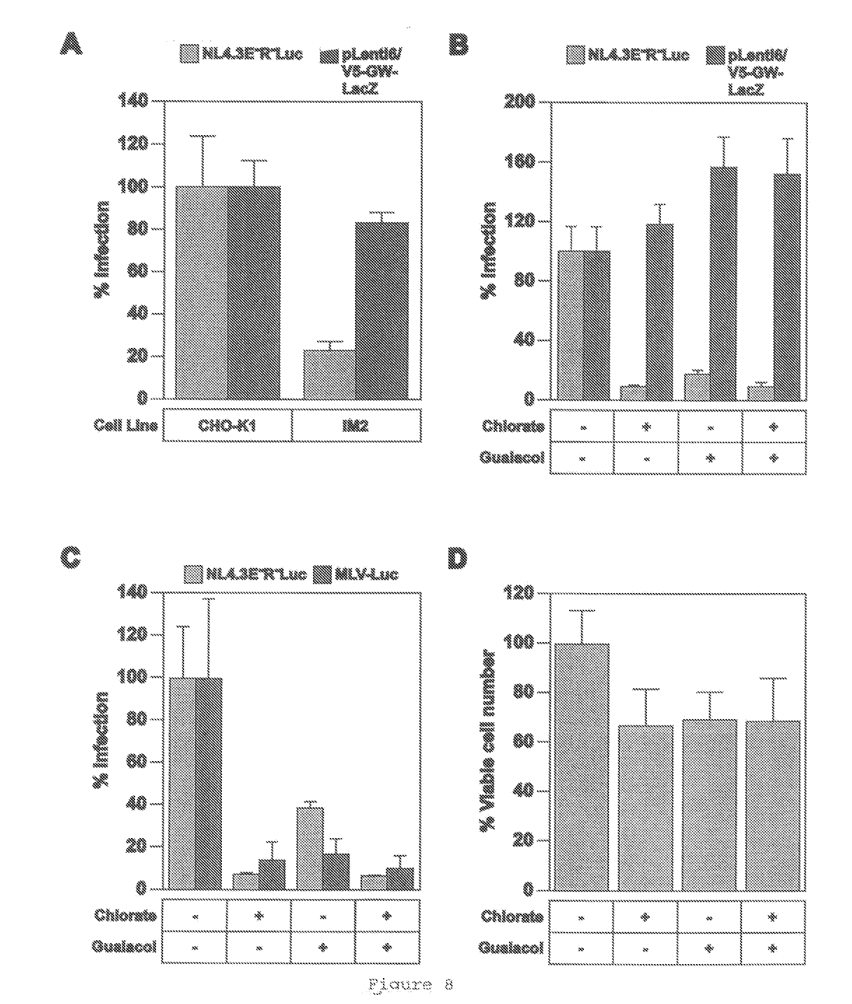

The sulfonation pathway affects HIV LTR-driven transcription: The previous experiments showed that the sulfonation pathway affects LTR-driven gene expression from newly acquired MLV, but not ASLV, proviruses. To test the influence of this pathway on HIV-1 LTR-driven gene expression, CHO-K1 and IM2 cells were challenged with either of two VSV-G pseudotyped HIV-1 vectors, one with luciferase expressed from the viral LTR and the other a SIN vector with β-galactosidase expressed from an internal CMV promoter. Reporter gene expression from the HIV-LTR was reduced 5-fold in IM2 versus CHO-K1 cells whereas that from the internal CMV promoter was the same in both cell types (FIG. 8A). Consistently, treatment of CHO-K1 cells with chlorate, guaiacol, or with both inhibitors resulted in 10-, 8-, and 12-fold reductions in HIV LTR-driven reporter gene expression, respectively. By contrast, internal CMV promoter-driven reporter gene expression was unaltered or was slightly enhanced by these treatments (FIG. 8B). Similar results were observed using human Jurkat T cells infected with VSV-G pseudotyped HIV or MLV viral vectors that express luciferase from the viral LTRs (FIG. 8C). Therefore as for MLV, HIV LTR-driven gene expression is also regulated by the cellular sulfonation pathway.

Discussion

Here we have presented multiple lines of evidence that the host cell sulfonation pathway influences retroviral infection by affecting a step during provirus establishment, one that modulates gene expression from the viral LTR promoter. First, insertional mutagenesis and genetic complementation studies identified PAPSS1 as a cellular gene that is important for MLV infection. Second, a similar defect was seen with cells treated with the PAPS synthetase inhibitor, chlorate, or with the sulfotransferase inhibitor, guaiacol. Third, inhibition of the sulfonation pathway had no impact on the levels of integrated MLV DNA but influenced downstream MLV LTR-driven gene expression from newly formed proviruses. Fourth, MLV was sensitive to inhibitors of the sulfonation pathway at time points up to that associated with maximal levels of viral DNA integration (Roe et al., EMBO. J. 12:2099-2108, 1993). Finally, the observations made with MLV held true for HIV-1, the causative agent of AIDS, since the sulfonation pathway also influenced LTR-driven transcription from that virus.

These data suggest that sulfonation may play an important role in the regulation of nuclear gene expression. Consistent with this, PAPSS1 localizes to the nucleus, which implies there is a requirement for high levels of PAPS, and by extension sulfonation, in the nucleus (Besset et al., FASEB J. 14:345-354, 2000). Thus, these studies have uncovered a heretofore unknown regulatory step of retroviral replication, one that is potentially important for HIV/AIDS therapy.

The data in this report are consistent with either one of two models. In the first model, the sulfonation pathway might influence viral DNA integration site specificity so that when this pathway is impaired, the virus is targeted to regions where the provirus is less transcriptionally competent. This model is consistent with the observation that viruses sensitive to the sulfonation pathway, HIV and MLV, both share a strong preference for integration into genes, although MLV shows a much stronger preference for integration near the viral promoter regions (Lewinski et al., PLoS Pathog. 2:e60, 2006; Mitchell et al., PLoS Biol. 2:E234, 2004; Schroder et al., Cell 110:521-529, 2002; Wu et al., Science 300:1749-1751, 2003). By contrast, ASLV, which is not influenced by this pathway, shows little or no preference for integration into genes (Mitchell et al., PLoS Biol. 2:E234, 2004; Narezkina et al., J. Virology 78:11656-11663, 2004).

In the second model, the sulfonation pathway might have no impact upon integration site specificity but, during integration or shortly thereafter, the sulfonation pathway might influence the nature of epigenetic modifications introduced onto the viral DNA. These modifications could, in turn, regulate the transcriptional competency of the provirus. Sulfonation induced changes in DNA methylation, histone acetylation, methylation or positioning are all potential processes which could affect the transcriptional activity of the provirus (Agbottah et al., Retrovirology 3:48, 2006; Pumfery et al., Current HIV Res. 1:343-362, 2003). Indeed the importance of epigenetic modifications in HIV transcription is apparent in a recent large scale analysis of HIV integration sites which revealed a positive correlation between integration and epigenetic modifications favoring transcription and a negative correlation with modifications that silence transcription (Wang et al., Genome Res. 17:1186-1194, 2007). We are currently performing experiments aimed at distinguishing between these two models.

The host cell sulfonation pathway involves a set of golgi and cytoplasmic sulfotransferases (SULTs) that transfer the sulfonate from PAPS to target substrates. In humans there are thirteen distinct cytosolic SULTs, arranged into three different families, and these enzymes are involved in the metabolism of steroids, bile acids, neurotransmitters, and xenobiotics (Gamage et al., Toxicol. Sci. 90:5-22, 2006). Golgi sulfotransferases are involved in sulfonating carbohydrates, generating the glycosaminoglycans (GAGs), heparan sulfate, chondroitin/dermatan sulfate, and keratan sulfate (Kusche-Gullberg et al., Current Opinion Struct. Biol. 13:605-611, 2003), as well as glycolipids (Strott, Endocr. Rev. 23:703-732, 2002). Two golgi tyrosylprotein sulfotransferases (TPST-1 and TPST-2) are responsible for sulfonation of tyrosine residues on proteins and peptides.

Tyrosyl sulfonation can have important regulatory effects on cell surface proteins including an influence on protein-protein interactions (Kehoe et al., Chem. Biol. 7:R57-61, 2000), as exemplified by the requirement for sulfonation of tyrosine residues at the amino-terminus of the CCR5 chemokine receptor for high affinity interaction with both its natural ligands, MIP-1a and MIP-1b, as well as with HIV-1 gp120 (Farzan et al., Cell 96:667-676, 1999). This entry effect seen previously is distinct from our observation that sulfonation also affects a post entry step coinciding with provirus establishment. Since inhibition of sulfonation can block HIV at multiple stages of the viral lifecycle, the cellular sulfonation pathway is an intriguing target for the development of novel antivirals.

Future work will be aimed at identifying the specific components of the sulfonation pathway that are critical for modulating MLV and HIV-1 infection. We expect that this information will help to uncover precisely how the sulfonation pathway regulates retroviral infection at a step coincident with provirus establishment and that influences the subsequent transcriptional competency of the provirus.

### Example 2

A clonal cell line (IM2) exhibiting ten-fold reduction in MLV-vector reporter gene expression was isolated from a population of insertionally mutagenized cells by multiple rounds of VSV-G pseudotyped MLV challenge and depletion of infected cells by magnetic sorting. Reverse transcription PCR identified PAPSS1 as the disrupted gene in IM2 cells. Expression of PAPSS1, and the related PAPSS2, complemented the MLV resistance phenotype of IM2 cells. PAPSS1 and PAPSS2 synthesize 3′ phosphoadenosine 5′ phosphosulfate (PAPS)—the high energy sulfate donor utilized in all cellular sulfonation reactions.

The role for the cellular sulfonation pathway in retroviral infection was confirmed by the use of chlorate, an inhibitor of PAPSS1 and 2, and guaiacol, an inhibitor of aryl sulfotransferases. Treatment of cells with either chemical inhibited both MLV and HIV vector reporter gene expression, while an ASLV vector was unaffected. Quantitative real time PCR analysis revealed that cellular sulfonation acts during or after the viral DNA integration step. Indeed, the timing of chlorate sensitivity is coincident with that of maximal viral DNA integration. Sensitivity to sulfonation inhibitors was mapped to the MLV and HIV LTRs. However, inhibition of the cellular sulfonation pathway affects expression from incoming newly acquired viruses but is not involved in transcriptional control of established proviral DNA. Therefore, sulfonation is most likely affecting either integration site selection or epigenetic modification of the viral DNA as the provirus is being established.

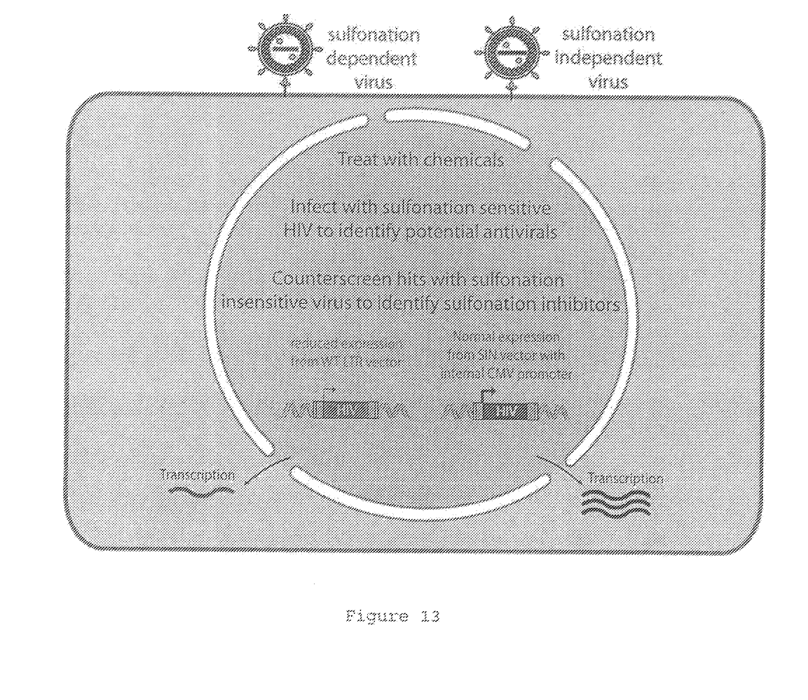

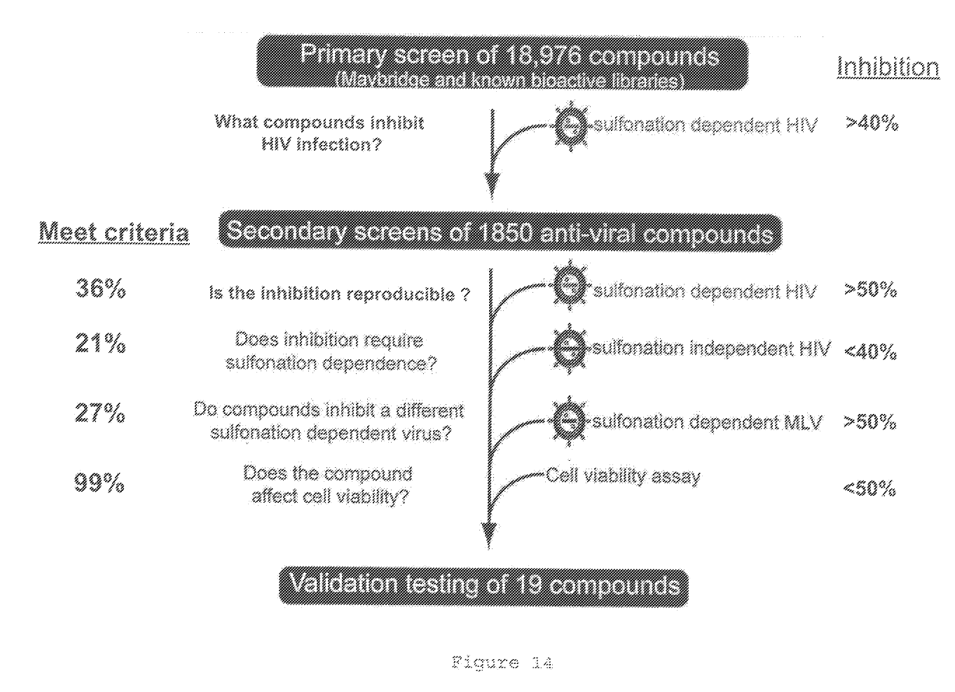

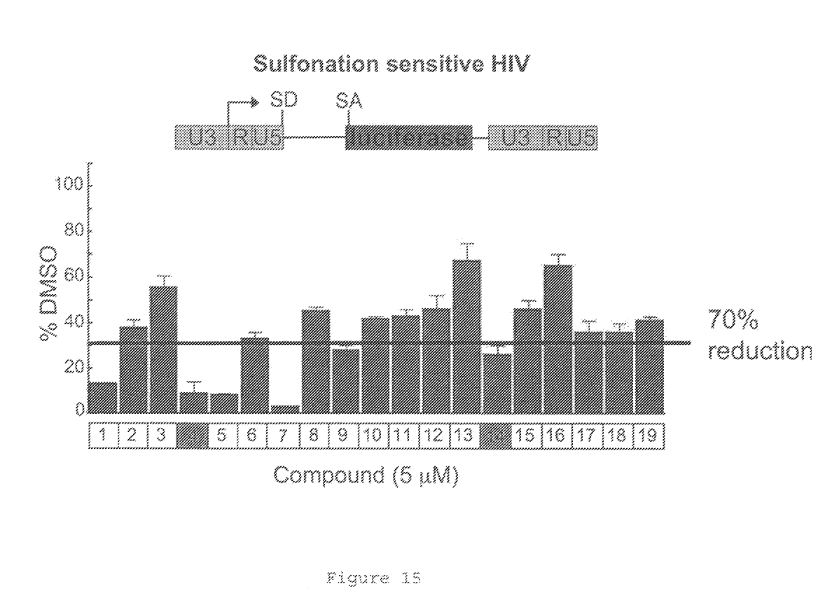

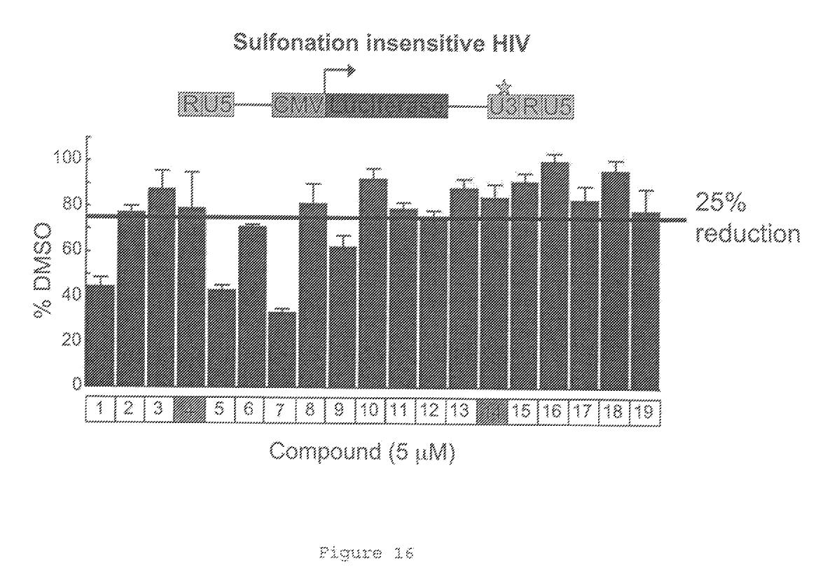

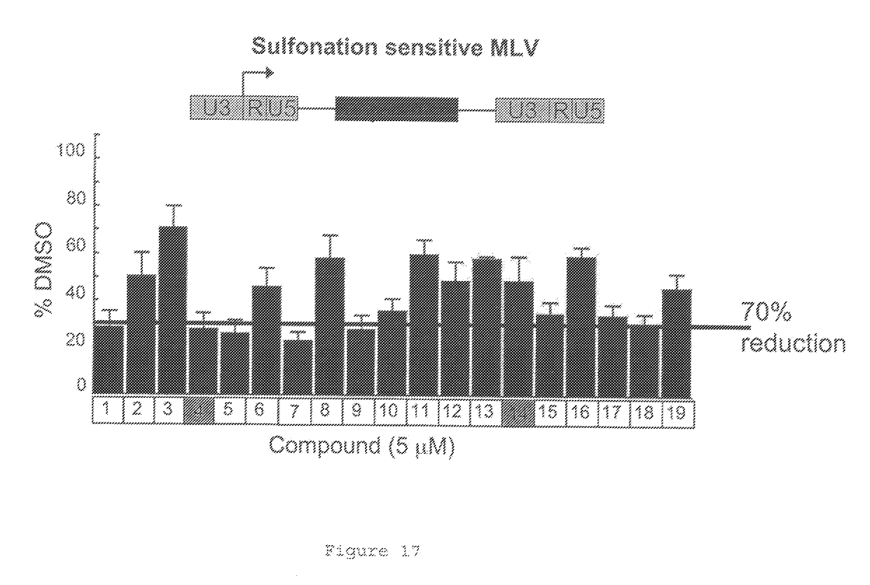

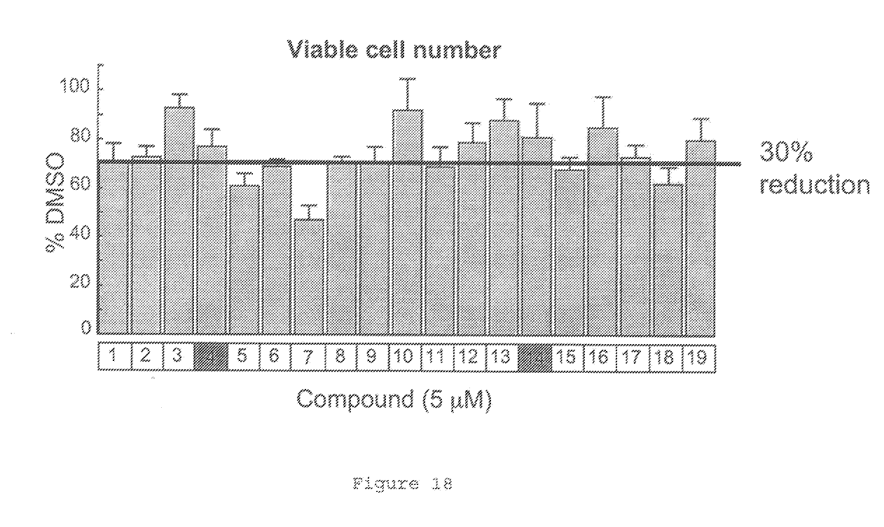

We have designed a high-throughput screening approach based on the sulfonation sensitive and insensitive forms of HIV and utilized it to identify small molecule inhibitors of sulfonation dependent HIV infection (FIG. 13). We have currently completed the primary and secondary screens of the Maybridge and known bioactive chemical libraries (FIG. 14) and have a number of potential compounds that appear to inhibit HIV infection in a sulfonation dependent manner (summarized in FIGS. 15-18).

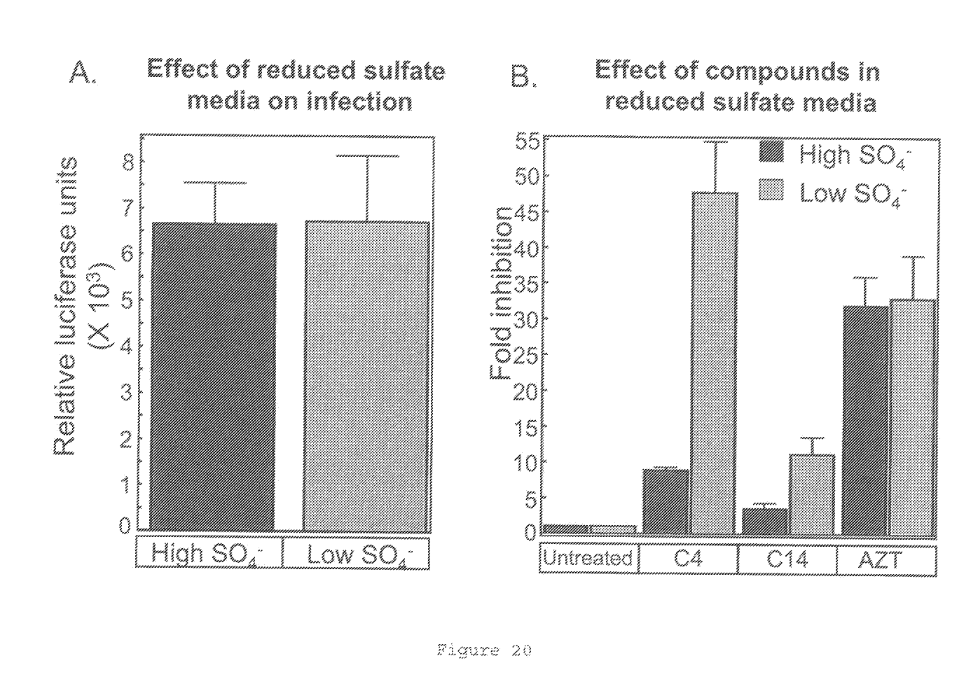

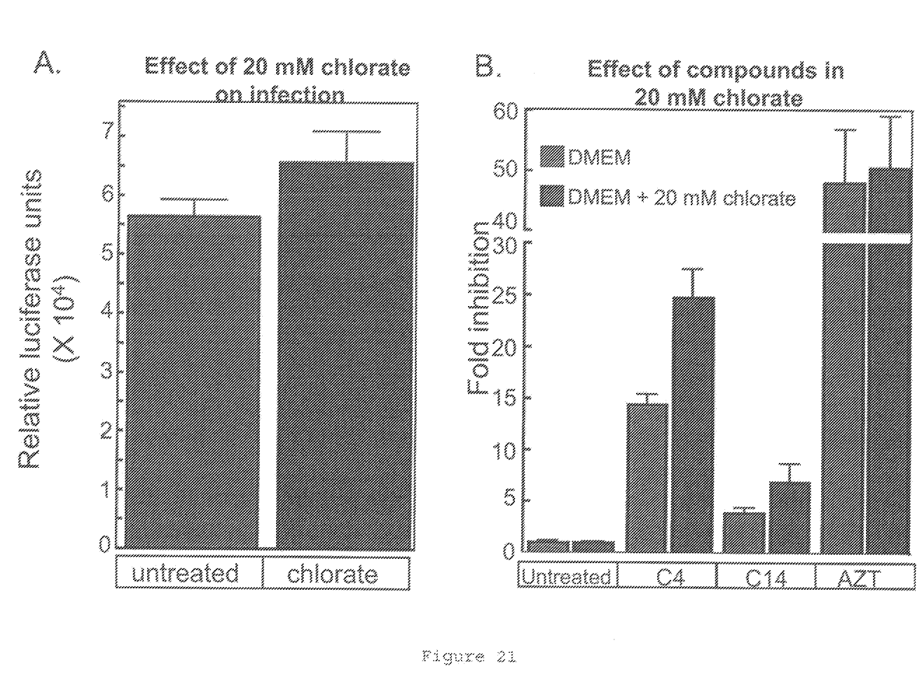

All of these compounds have been tested against sulfonation dependent and independent forms of HIV and MLV, as well as for effects on cell viability across a wide concentration range. Consistent with their proposed effect on sulfonation, two of these compounds (C4 and C14) with the highest activities preferentially inhibited gene expression from newly acquired, but not established, proviruses (FIG. 9). They also exhibited synergy with known sulfonation inhibitors and further reduced infection when cells were grown in reduced sulfate-containing media (FIGS. 20 and 21).

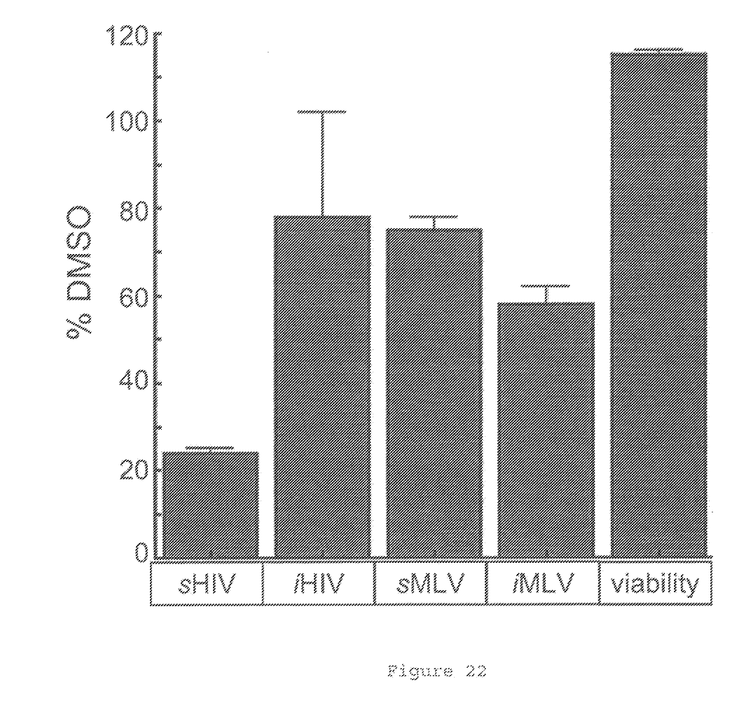

Currently we are pursuing structure activity relationship (SAR) analysis on the two most highly effective compounds against both HIV and MLV, as well as the one compound identified that specifically inhibited only sulfonation dependent HIV infection (FIG. 22). These studies have identified novel candidate inhibitors of the cellular sulfonation pathway, compounds that will be useful for further characterizing the role played by this cellular pathway in retroviral infection. Since these compounds are also important leads for developing novel HIV/AIDS therapies aimed at targeting the cellular sulfonation pathway, we are currently pursuing SAR analysis on the most active compounds.

SAR analysis will include but will not be limited to: (1) sourcing of structurally similar derivatives of the three lead compounds for testing in the assays described herein; (2) analysis of the assay results to determine structural attributes of lead pharmacophores that are critical for maintaining or improving activity; (3) design and synthesis (and/or sourcing, if available and affordable derivatives can be purchased) of a focused SAR library based on the information obtained from (2) to optimize activity against viral infection, (4) identification of a highly active drug lead for advanced studies.

1. A method of screening test agents as inhibitors of human immunodeficiency virus (HIV) replication, comprising the step of
(a) determining whether the test agent is a sulfonation inhibitor, wherein if the test agent is a sulfonation inhibitor, then the test agent is a suitable inhibitor of HIV replication.

2. The method of claim 1 additionally comprising, the following step:
(b) determining whether the test agent is an inhibitor of 3′-phosphoadenosine 5′-phosphosulfate synthase 1 (PAPSS1).

3. The method of claim 1 additionally comprising the following step:
(b) determining whether the test agent is an inhibitor of at least one sulfotransferase.

4. A method of screening test chemicals or compounds for inhibition of HIV replication, comprising the steps of
(a) exposing a test chemical or compounds to a cell;
(b) exposing a first and a second HIV vector to the cell, wherein the first HIV vector is sensitive to the cell's sulfonation pathway and the second HIV vector is insensitive to the cell's sulfonation pathway and wherein both HIV vectors comprise genes encoding reporter molecules; and
(c) examining the result of steps (a) and (b), wherein a test chemical or compound that interferes with reporter gene expression from the first, but not the second, HIV vector, is a suitable inhibitor of HIV replication.

5. The method of claim 4 wherein the test chemical interferes with the function of a sulfonation-regulated effector of HIV gene expression.

6. The method of claim 4 wherein the cell is a mammalian cell.

7. The method of claim 4 wherein the cell is selected from the group consisting of HEK293 cells, Jurkat cells, other human T-cell lines, human acute monocytic leukemia cell lines (THP-1), other human macrophase/monocyte cell lines, primary T lymphocytes, and primary macrophage/monocytes.

8. A method of treating an HIV-infected individual to reduce HIV replication comprising the step of treating the individual with an effective amount of sulfonation inhibitor.

9. The method of claim 7 wherein the inhibitor is an inhibitor of PAPSS1.

10. The method of claim 7 wherein the inhibitor is an inhibitor of at least one sulfotransferase.

11. The method of claim 4 wherein the reporter gene of the sulfonation insensitive vector is β-galactosidase.

12. The method of claim 4 wherein the first and second HIV vectors are pseudotyped with the vesicular stomatitis virus glycoprotein.

13. The method of claim 4 wherein the expression of reporters is measured by chemiluminescent assay.

14. The method of claim 4 wherein the sulfonation insensitive vector is PLenti6/V5-GW/lacZ.

15. The method of claim 4 additionally comprising the following step:
(d) exposing a third and fourth retroviral vector to the cell, wherein the third vector has long terminal repeats (LTRs) that are sensitive to sulfonation pathway inhibition and the fourth vector has LTRs that are not sensitive to sulfonation pathway inhibition and wherein both the third and fourth retroviral vectors comprise genes encoding reporter molecules, and
(e) examining the results of step (d), wherein test chemical or compound that interferes with reporter gene expression for the third, but not the fourth, retroviral vector is a suitable inhibitor of HIV replication.

16. The method of claim 15 wherein the third retroviral vector is selected from the group comprising HIV and murine leukosis virus (MLV).

17. The method of claim 15 wherein the fourth retroviral vector is selected from the group comprising avian sarcoma and leukosis virus (ASLV).

18. The method of claim 4 additionally comprising the following step:
(d) measuring the cell viability.

19. The method of claim 4 wherein the test chemical and HIV vectors are exposed to the cells sequentially.

20. The method of claim 4 wherein the first and second HIV vectors and test chemical are exposed to the cells concurrently.

21. (canceled)

22. (canceled)

23. (canceled)

24. (canceled)

25. (canceled)

26. (canceled)
